(19) United States
(12) Patent Application Publication
(10) Pub. No.: US 2024/0393241 A1
(43) Pub. Date: Nov. 28, 2024

(54) ZWITTERIONIC SURFACES FOR LOCALIZED SURFACE PLASMON RESONANCE

(71) Applicant: THE TRUSTEES OF INDIANA UNIVERSITY, Bloomington, IN (US)

(72) Inventors: Rajesh SARDAR, Avon, IN (US); Barry B. MUHOBERAC, Indianapolis, IN (US); Adrianna N. MASTERSON, Indianapolis, IN (US); Sumon HATI, Indianapolis, IN (US)

(21) Appl. No.: 18/691,513

(22) PCT Filed: Sep. 7, 2022
(86) PCT No.: PCT/US2022/042805

§ 371 Date: Mar. 13, 2024

Related U.S. Application Data
(60) Provisional application No. 63/247,474, filed on Sep. 23, 2021.

(51) Int. Cl. G01N 21/552; B82Y 15/00; B82Y 20/00; C12Q 1/6825; G01N 33/543; G01N 33/58

(52) U.S. Cl. CPC G01N 21/554; B82Y 15/00; B82Y 20/00; C12Q 1/6825; G01N 33/5438; G01N 33/582

(57) ABSTRACT

Disclosed herein is a plasmonic molecular device consisting photoisomerizable molecular switch-tethered gold triangular nanoprisms and methods for using such device for the detection of biomarkers. The molecular device exhibits unprecedentedly large localized surface plasmon resonance shifts during the photoisomerization of molecular switches. The fabricated molecular device with zwitterionic structure has been utilized to develop adaptable nanoplasmonic biosensor for ultrasensitive. highly specific and programmable detection of microRNAs and proteins from patient biofluids.

### CROSS REFERENCE TO RELATED APPLICATIONS

This application claims priority to the following: U.S. Provisional Patent Application No. 63/247,474 filed on Sep. 23, 2021, the disclosure of which is expressly incorporated herein.

### GOVERNMENT LICENSE RIGHTS

This invention was made with government support under CBET1604617 awarded by National Science Foundation. The Government has certain rights in the invention.

### BACKGROUND

The SARS-COV-2 (COVID-19) pandemic has shown how ill-prepared modern bioanalytical assays are in combating deadly infectious diseases. This is, in part, due to the major bottlenecks existing in univariant “gold standard” bioanalytical techniques (RT-PCR, microarray, sequencing, ELISA, etc.), which almost exclusively operate in a discrete format unable to assay different types of biomolecules using single instrument.

MicroRNAs (small non-coding RNAs with a 17-25 nucleotide sequence) and lncRNAs (larger than 200 bases) are involved in many cellular processes including gene transcription, mRNA translation, and protein function. Their expression levels are altered in diseases such as cancer, viral infection, neurodegeneration, and autoimmune diseases and represent key diagnostic biomarkers. Quantitative real time-polymerase chain reaction (qRT-PCR) and genotyping are widely used to quantify RNAs for basic biomedical research and clinical diagnostics. However, these techniques cannot be used to analyze proteins, vital in enzymatic reactions and as structural components, where abnormal behavior causes many diseases. Therefore, for a comprehensive picture, multiple techniques must be run in parallel.

Western blot, ELISA, and surface plasmon resonance are common protein assay technique. However, all of these techniques suffer from serious problems, including the need for labelling and pre-amplification, exhaustive preparation/purification steps, and low specificity and sensitivity. Also, these univariant methods only detect one type of biomolecule.

Accordingly there is a need for analytical methodologies multiplex (i.e., detecting several types of biomarkers using the same instrument setup) to allow for high-throughput screening in point of care facilities. Beyond lowering costs and time lag, multiplexing assays for a disease mitigates false responses to maximize precision and accuracy.

As disclosed herein, to overcome current challenges in bioanalytical assays and create more accurate, reliable and reproducible early disease diagnostics, an entirely new, and highly adaptable nanoplasmonic biosensor (biosensing chip and assay switching protocol) is provided that is capable of high sensitivity and simultaneous measurement of three important classes of biomarkers, i.e., microRNAs, long non-coding RNAs (lncRNAs), and proteins using a single UV visible spectrophotometer (multiplexing). Importantly, the biosensor uses an identical structural motif to detect these diverse biomolecules, programming detection by simply exchanging attached receptor molecules using light (FIG. 1A).

### SUMMARY

Modulating optoelectronic properties of nanostructures tethered with light-responsive molecular switches by their conformational change in solid-state is fundamentally important for advanced nanoscale-device fabrication. All the systems reported thus far describe only changes in the optical response of nanostructures in solution phase. As disclosed herein a new solid-state design approach is provided.

The biosensors of the present disclosure utilize localized surface plasmon resonance (LSPR) properties of chemically-synthesized gold triangular nanoprisms (Au TNPs) coupled with the molecular dipole of zwitterionic surfaces to enhance plasmonic response and increase biosensor efficiency and sensitivity. Accurate, reliable, fast, sensitive, selective, and reproducible detection of a variety of disease-related biomarkers is key to point-of-care (POC) clinical diagnostics. As disclosed herein the present device provides a construct that is adaptable, multiplexing (assaying multiple biomarkers of a same type, or those with different expression levels such as oncogenic and tumor suppressor microRNAs), multimodal (detection via LSPR-and SERS-based techniques with same biosensing chips), and provides high-throughput (ability to assay hundreds of samples simultaneously), which will unprecedentedly improve high accuracy disease diagnosis at POC. In particular, these novel biosensors should provide a more accurate and precise assay due to same sample, regeneratively testing capabilities for different biomarkers (adaptability), e.g., first as an microRNA assay, then regenerating biosensors to measure lncRNAs, and finally regenerating again to assay proteins, all using the same device and identical signal output. Using this approach, the possibility of high false positive and negative responses is minimized due to less variability in the assay. Biosensor regeneration is also an important aspect in lowering costs, particularly important in low-income countries. This strategy is less labor intensive, offers excellent protocol verification, and offers good biosensor calibration and standardization. Furthermore, more than one biomarker is assayed with only one instrument needed, reducing time-lag dramatically improving diagnosis by faster data correlation.

In accordance with one embodiment an adaptable nanoplasmonic biosensor is provided for use in the detection of target proteins and nucleic acids in a biological sample. The biological sample can be a bodily fluid including for example blood, serum, plasma, urine or saliva, either as a crude sample, or as a fractionated component of the sample or a purified component of the crude sample. In one embodiment the biosensor comprises a localized surface plasmon resonance (LSPR) chip having an affixation surface and a functional surface, a functionalized solid support and a plurality of LSPR antennae polymers that comprise a light inducible isomerizable compound, wherein the affixation surface of the LSPR chip is covalently linked to said solid support, and the LSPR antennae are linked to said functional surface of the LSPR chip. In one embodiment the LSPR chip is a metal comprising triangular nanoprism, wherein the metal is selected from the group consisting of gold, silver, copper, palladium, aluminum, or a combination thereof. In one embodiment the LSPR chip is a silver or gold triangular nanoprism, wherein the LSPR chip is covalently linked to the solid support via a plurality of spacer molecules that comprise a poly-ethylene glycol moiety, an alkyl moiety, or a combination thereof. In one embodiment an adaptable nanoplasmonic biosensor is provided wherein said LSPR antennae is linked to a functional surface of the LSPR chip via a plurality of spacer molecules that comprise a poly-ethylene glycol moiety, an alkyl moiety, or a combination thereof, optionally wherein the LSPR chip is a gold triangular nanoprism (Au TNP) and said spacer molecules comprise a first end bound to the LSPR antennae and a second end comprising a functional group, optionally a thiol, that forms a covalently bond to group located on the functional surface of the LSPR chip.

In one embodiment the biosensor of the present disclosure comprises a gold triangular nanoprism (Au TNP) linked to light transparent solid support via a plurality of spacer molecules, wherein the spacer molecules comprise a first end bound to the solid support and a second end comprising a functional group, optionally a thiol, that forms a covalently bond to a group located on the affixation surface of the LSPR chip. In one embodiment the Au TNP has an average edge-length of between 30 and 50 nm, optionally having an average edge-length of about 42 nm and 8 nm height (AFM analysis) that is attached onto thiolated silane-functionalized solid support that is substantially transparent to electromagnetic radiation having a wavelength between 100 nm and 700 nm. In one embodiment the solid support is glass.

In one embodiment the biosensor of the present disclosure comprises light inducible isomerizable compounds that form a zwitterion upon exposure to UV light and convert back a non-zwitterion form upon exposure to visible light, wherein said light inducible isomerizable compounds are covalently linked to said functional surface of the LSPR chip via a plurality of alkylthiolate spacer molecules, optionally wherein the alkylthiolate spacer molecules are nonanethiol or undecanethiol spacer molecules. In one embodiment the light inducible isomerizable compound has the general structure of

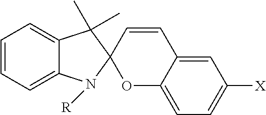

wherein R is selected from the group consisting of —(CH₂)ₙN⁺(CH₃)₃, wherein n is an integer selected from 1 to 6, and
X is —NO₂, —CONH₂ or —COOH. In one embodiment the biosensor of the present invention comprises a light inducible isomerizable compound having the structure of

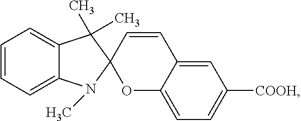 [SMILES: CN1c2ccccc2C(C)(C)C12C=Cc1cc(C(=O)O)ccc1O2]

and in one embodiment the LSPR antennae comprises the structure:

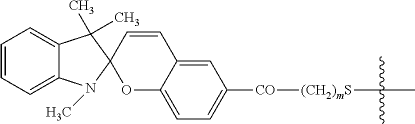

wherein m is an integer selected from 9 to 13, optionally wherein m is 11, i.e., a spiropyran-undecanethiol (SP-UT). In one embodiment the biosensor further comprises a plurality of undecanethiol (UT) polymers linked to the functional surface of the LSPR chip and interspersed between spiropyran-undecanethiol (SP-UT). In one embodiment the ratio of SP-UT to UT polymers linked to the functional surface of the LSPR chip is selected from the ratios of 65%:35%, 70%:30%, 75%:25%, 80%:20% and 85%:15%. In one embodiment the biosensor comprises a 75%:25% mixture of SP-UT and UT linked to the functional surface of the LSPR chip.

In one embodiment, the biosensor of the present disclosure, comprising a light-inducible reversible conformational change of spiropyran (SP)-merocyanine (MC) covalently attached to gold triangular nanoprisms (Au TNPs) via alkylthiolate self-assembled monolayers, can be used to produce a large localized surface plasmon resonance response (˜24 nm). This shift is consistent with the increase in thickness of the local dielectric shell-surrounded TNPs and perhaps short-range dipole-dipole (permanent and induced) interactions between TNPs and the zwitterionic MC form. Accordingly, this adaptable nanoplasmonic biosensor can be utilized for ultrasensitive, highly specific and programmable detection of microRNAs and proteins at sub-attomolar concentrations in a patient's biological bodily fluid, all while using an identical structural motif. Taken together, the presently disclosed photochromatic isomerization of SP-MC in solid-state is not only important for examining the dipole-dipole interactions across the nanostructure-organic molecule interface, but also the TNP-MC structural motif represents a multifunctional super biosensor with the potential to expand clinical diagnostics through simplifying biosensor design and providing highly accurate disease diagnosis.

In one embodiment a method of detecting the presence of a first and second analyte in a biological sample through the use of a single device utilizing an identical signal output for the detection of both the first and second analyte is provided. In one embodiment the method comprises
a) providing any of the nanoplasmonic biosensors disclosed herein and employing a light-induced reversible conformational change of spiropyran (SP)-merocyanine (MC) covalently attached to metal nanoparticals, optionally gold triangular nanoprisms (Au TNPs) via alkylthiolate self-assembled monolayers;
b) exposing the biosensor to UV light to induce photoisomerization of said isomerizable compound and produce the zwitterion form of said compound;
c) contacting the zwitterion form of said compound with a first ligand that specifically binds to said first analyte, wherein the first ligand electrostatically binds to the zwitterion form of said compound to form a first ligand complex;
d) contacting said first ligand complex with a biological sample;
e) conducting LSPR analysis of said nanoplasmonic biosensor after step d);
f) optionally rinsing said first ligand complex with a buffer to promote charge screening to disrupt binding of the first ligand to the zwitterion form of said compound;
g) exposing the biosensor to visible light (>450 nm) to regenerate the non-zwitterion form of said compound and rinsing said biosensor to remove unbound material;
h) exposing the biosensor to UV light (<450 nm) to induce photoisomerization of said isomerizable compound and produce the zwitterion form of said compound;
i) contacting the zwitterion form of said compound with a second ligand that specifically binds to said second analyte, wherein said second analyte electrostatically binds to the zwitterion form of said compound to form a second ligand complex;
j) contacting said second ligand complex with a biological sample;
k) conducting LSPR analysis of said nanoplasmonic biosensor after step j). In one embodiment the non-zwitterion form of said compound comprises the structure of
of

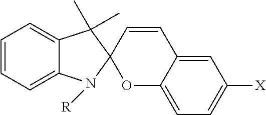

wherein R is selected from the group consisting of —(CH₂)ₙN⁺(CH₃)₃, wherein n is an integer selected from 1 to 6, and
X is —NO₂ or —COOH; and
   said zwitterion form of said compound comprises the structure of

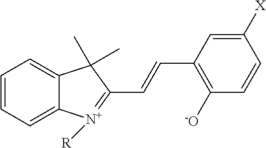

wherein R is selected from the group consisting of —(CH₂)ₙN⁺(CH₃)₃, wherein n is an integer selected from 1 to 6, and
X is —NO₂ or —COOH. In one embodiment the biosensor used in the method comprises a gold triangular nanoprism (Au TNP), said light inducible isomerizable compounds are covalently linked to said functional surface of said Au TNP via a plurality of undecanethiol spacer molecules, further wherein the functional surface of the Au TNP also comprises a plurality of undecanethiol (UT) polymers linked to said functional surface of the LSPR chip. In one embodiment the steps of conducting an LSPR analysis comprises measuring an absorption spectrum of the LSPR antenna, the absorption spectrum having a peak wavelength; and determining the presence or quantity of the first and second analyte in said sample based on the peak wavelength. In one embodiment the method of detecting the presence of the first analyte in said sample comprises measuring a first absorption spectrum of the LSPR antenna after step c) and measuring a second absorption spectrum of the LSPR antenna after step d) and determining the difference between the peak wavelength of the first and second measured absorption spectrum of the LSPR antenna; and detecting the presence of the second analyte in said sample comprises measuring a third absorption spectrum of the LSPR antenna after step i) and measuring a fourth absorption spectrum of the LSPR antenna after step j) and determining the difference between the peak wavelength of the third and fourth measured absorption spectrum of the LSPR antenna.

In one embodiment of the method of detecting the presence of a first and second analyte, the first analyte is a protein, the second analyte is a nucleic acid, the first ligand is a protein receptor that specifically binds to said protein and the second ligand is a nucleic acid sequence that specifically binds to said nucleic acid.

In one embodiment of the method of detecting the presence of a first and second analyte is provided, wherein the first analyte is a first RNA, the second analyte is a second RNA, the first ligand is a first nucleic acid sequence that specifically binds to said first RNA and the second ligand is a nucleic acid sequence that specifically binds to said second RNA, wherein the first and second RNAs have difference nucleic acid sequences.

In one embodiment the methods of the present disclosure are used to detect the presence of a first, second and third analyte, wherein steps g)-k) are repeated wherein the zwitterion form of said compound is contacted with a third ligand that specifically binds to said third analyte.

### BRIEF DESCRIPTION OF THE DRAWINGS

FIGS. 1A-1D: FIG. 1A is a schematic illustration of functionalized Au TNPs as a solid-state biosensor and the method of detecting microRNAs and proteins using the biosensor as shown in steps A-F. Au TNPs were attached onto thiolated silane-functionalized glass coverslip and the TNP surface was modified with a 75:25% ratio of SP-UT and UT SAMs (labeled as step A). Then the coverslip was exposed to UV light to create the MC-UT form (labeled as step B). The coverslips were incubated in PBS buffer containing either -ssDNA or receptor protein. -ssDNAs (labeled as step C) or protein (labeled as step D) electrostatically are adsorbed onto the zwitterionic MC surface to complete the “nanoplasmonic biosensor”. The biosensor was incubated overnight in either human plasma or urine supplemented with microRNA or protein. Here, -ssDNA (receptor) has the complementary base pair for the target microRNA (analyte) (labeled as step E), and receptor protein specifically binds to analyte protein (labeled as step F). Receptor molecules and bound analytes are dissociated by light exposure ((>450 nm) and new receptors and analytes can bind completing the cycle and making the biosensor adaptable. Au TNPs were functionalized with different chain length alkylthiols. The distance was determined between the Au atom and terminal H of CH₃ group of alkanethiolate SAMs (slope=20.88815, R²=0.992). Geometric optimization molecular distance between Au atom and terminal H of SP-UT (FIG. 1B) and MC-UT (FIG. 1C) are shown. FIG. 1D is a graph presenting the Δλ_{LSPR} value as a function of dielectric shell thicknesses.

FIGS. 2A-2I provides analysis of the constructed Au TNPs. FIGS. 2A and 2B provide representative scanning electron (FIG. 2A) and atomic force (FIG. 2B) microscopy images of Au TNPs. FIG. 2C: UV-visible extinction spectra of Au TNPs before (1, λLSPR=800.1 nm), after functionalization with 100% UT (0% SP-UT) (2, λLSPR=828.1 nm), and with 100% SP-UT (0% UT) (3, λLSPR=868.2 nm). FIG. 2D: Bar graph representation of ΔλLSPR (nm) for Au TNP functionalization with different percentages of SP-UT (remaining percentage is UT spacer). FIG. 2E: UV-visible extinction spectra of Au TNPs before (1, λLSPR=801.0 nm), after functionalization with 75:25% SP-UT:UT (SP-UT/UT) SAMs (2, λLSPR=864.1 nm), and after exposure to UV light (3, λLSPR=888.3 nm). FIG. 2F: Bar graph representation of ΔλLSPR (nm) for transformation of 75:25% SP-UT to 75:25% MC-UT form. FIG. 2G: Water drops on SP-UT:UT SAM-modified surface (top, θ=94°) and after UV light irradiation (bottom, θ=82.3°). FIG. 2H: The λLSPR position of 75:25% SP-UT:UT SAM-modified nanosystem upon cycling between exposure to UV and Visible light. Exposure to UV light caused SP to convert to the MC form, while visible light exposure caused a transition of MC to SP. (FIG. 2I UV-visible extinction spectra showing reversibility between SP-UT:UT SAM and MC-UT:UT after the first (SP-UT:UT, 1, λLSPR=864.0 nm and MC-UT:UT, 1, λLSPR=888.7 nm) and the tenth (SP-UT:UT, 3 dashed, λLSPR=865.0 nm and MC-UT:UT, 4 dashed, λLSPR=890.1 nm) cycle. All extinction spectra were collected in air.

FIG. 3A-3I: FIG. 3A displays a UV-visible extinction spectra of MC-UT:UT SAM-modified TNPs before (1, λ_{LSPR}=888.9 nm), after incubation in 10 μM -ssDNA-10b in PBS buffer (2, λ_{LSPR}=910.3 nm), and followed by incubation in 100 nM microRNA-10b in 10% human plasma (3, λ_{LSPR}=925.1 nm). FIG. 3B: UV-visible extinction spectra of TEA-passivated Au TNPs (1, λLSPR=799.1 nm) after UV light irradiation (2, λLSPR=797.0 nm) for 10 min, followed by Visible light irradiation (3, λLSPR=797.0) for 10 min. All extinction spectra were collected in air. FIG. 3C: An adsorption isotherm of -ssDNA-10b on MC-UT:UT SAM-modified surface. FIG. 3D: Calibration curves of microRNA-10b (top curve) and microRNA-145 (bottom curve) in human plasma in the range of 100 nM to 100 aM concentrations. FIG. 3E: Bar graph representation of microRNA-10b specificity test: MC-UT:UT SAM-modified TNPs (2LSPR=887.0 nm), incubation in 10 μM -ssDNA-10b (λ_{LSPR}=908.4 nm), and finally incubation in a mixture of 100 nM of each microRNA-145, 143, 490-5p, 96 solution (λ_{LSPR}=909.8 nm). FIG. 3F: UV-visible extinction spectra of MC-UT:UT SAM-modified TNPs before (1, λ_{LSPR}=885.0 nm), after incubation in 100 ng/ml anti-NuMA solution in PBS buffer (2, λ_{LSPR}=916.4 nm), and finally after incubation in 100 nM NuMA in 10% human urine (3, λ_{LSPR}=931.2 nm). FIG. 3G: An adsorption isotherm of anti-NuMA on a MC-UT:UT SAM-modified surface. FIG. 3H: Calibration curve of 100 nM to 100 aM NuMA in urine. FIG. 3I The bar graph of NuMA specificity test: MC-UT:UT-SAM modified TNPs (λ_{LSPR}=887.0 nm), after adsorption of anti-NuMA (λ_{LSPR}=918.4 nm), and then incubation of the biosensor in 100 nM PSA solution in 10% urine (λ_{LSPR}=920.8 nm). After each incubation, biosensors were rinsed with RNase free water and then extinction spectra were collected.

FIGS. 4A & 4B: FIG. 4A provides a schematic diagram representing adaptability of the nanoplasmonic biosensor to detect both microRNA-10b and NuMA protein. FIG. 4B provides a Bar graph representation of adaptability of the biosensor to detect both microRNA-145 and NuMA protein: SP-UT/UT (lane 1, λLSPR=863.0 nm), MC-UT/UT (lane 2, λLSPR=887.4 nm), anti-NuMA (lane 3, λLSPR=918.8 nm), 100 nM NuMA (lane 4, λLSPR=936.1 nm), SP-UT/UT (lane 5, λLSPR=864.2 nm), MC-UT/UT (lane 6, λLSPR=887.4 nm), -ssDNA-145 (lane 7, λLSPR=906.5 nm), and 100 nM microRNA-145 (lane 8, λLSPR=920.9 nm). After each incubation, biosensors were rinsed with RNase free water and then extinction spectra were collected.

FIGS. 5A-5F: Quantification of microRNA-10b (FIG. 5A), microRNA-145 (FIG. 5B), and NuMA (FIG. 5C) in ten bladder cancer patient plasma samples (metastatic, MT) and ten healthy plasma samples (normal control, NC). The p-values were determined by paired t-test: ***p<0.0002 and ****p<0.0001. FIG. 5D: Receiving operative characteristic curve of microRNA-10b (1), microRNA-145 (2), and NuMA (3) of MT bladder cancer patients and NC samples. FIG. 5E: Schematic representation of protocol verification in which microRNA-10b was quantified using the biosensor, the biosensor was regenerated, and then the proficiency was measured using a known concentration of microRNA-10b. FIG. 5F: Bar graph representation of confirmation test: mixed SP-UT and UT (λLSPR=864.4 nm), MC-UT and UT (λLSPR=890.2 nm), -ssDNA-10b (λLSPR=913.0 nm), patient sample MT-10 microRNA-10b quantification (λLSPR=920.5 nm), mixed SP-UT and UT (λLSPR=866.3nm), MC-UT and UT (λLSPR=891.5 nm), -ssDNA-10b (λLSPR=912.9 nm), and 100 nM microRNA-10b (λLSPR=928.2 nm).

FIG. 6: UV-visible extinction spectra of mixed MC-UT:UT-SAM modified Au TNPs (1, λ_{LSPR}=888.0 nm), after incubation in a 10.0 μM -ssDNA-145 solution (2, λ_{LSPR}=907.5 nm), and finally incubation in a 100.0 nM microRNA-145 in 10% human plasma (3, λ_{LSPR}=922.0 nm).

FIGS. 7A & 7B: Calibration curves of microRNA-10b (FIG. 7A) and microRNA-145 (FIG. 7B) in PBS buffer in the 100 nM to 100 aM concentration range for binding to appropriate -ssDNA attached to the mixed SP-UT:UT SAM-modified Au TNPs.

FIGS. 8A & 8B: Experimentally determined SERS spectra (top) and TDDFT calculated Raman spectra (bottom) of mixed SAMs of SP-UT:UT (FIG. 8A) and MC-UT:UT (FIG. 8B). In SP-UT:UT SAM, the vibrational modes in red font identify Raman stretches that match with Ivashenko et al., Langmuir 2013, 29, 4290-4297.10 The stretches in blue font identify Raman vibrational modes that match with TDDFT calculations (Table 1). In MC-UT:UT SAM, the Raman stretches in red font identify vibration modes that match with Ivashenko et al., Langmuir 2013, 29, 4290-4297. The vibration modes in blue font identify the Raman stretches that match with TDDFT calculations (Table 2).

### DETAILED DESCRIPTION

### Abbreviations

LSPR is an abbreviation for localized surface plasmon resonance.
“Au TNPs” is an abbreviation for gold triangular nanoprisms.
SAM is an abbreviation for self-assembled monolayers.
SP is an abbreviation for spiropyran.
MC is an abbreviation for merocyanine.
UT is an abbreviation for undecanethiol.

### Definitions

In describing and claiming the methods, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein the terms “effective amount” or “therapeutically effective amount” of a compound refers to a nontoxic but sufficient amount of the compound to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “subject” means an animal including but not limited to, humans, domesticated animals including horses, dogs, cats, cattle, and the like, rodents, reptiles, and amphibians.

As used herein the term “LSPR analysis” defines of process of measuring the resonant oscillation of conduction electrons at the interface between negative and positive permittivity material stimulated by incident light in both the presence and absence of a sample, and comparing the resonant oscillation detected from the two measurements. The detection of the resonant oscillation can be conducted through optical-based sample analyses including localized surface plasmon resonance (LSPR) and surface-enhanced Raman scattering (SERS).

### Embodiments

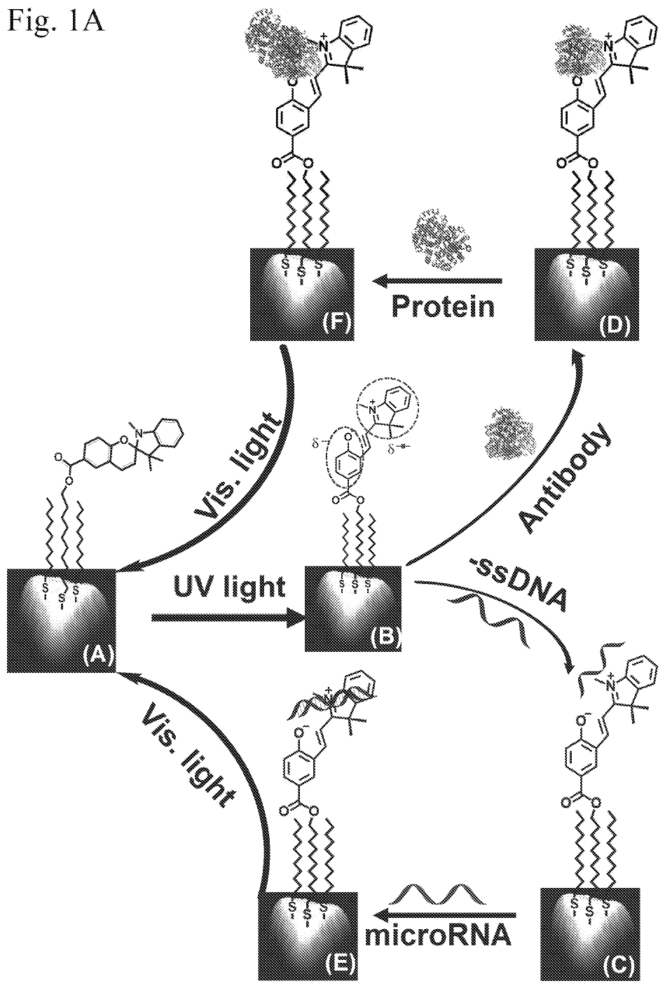

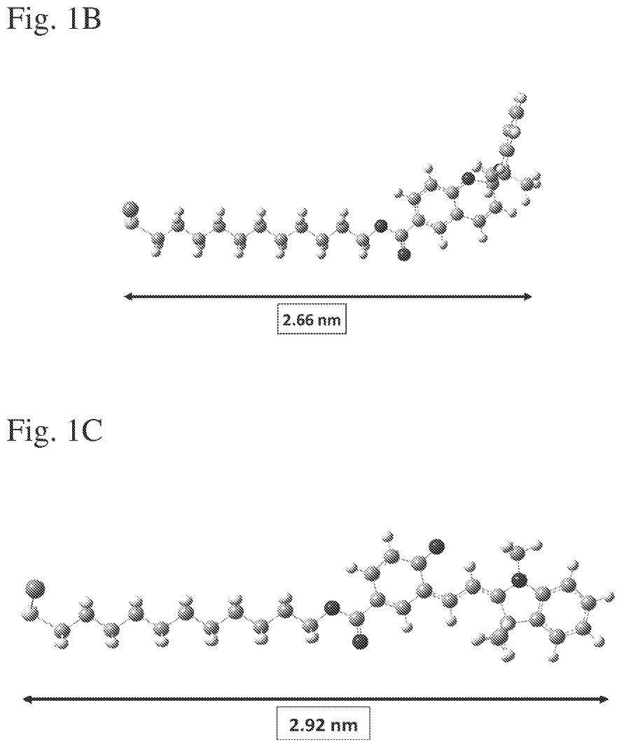

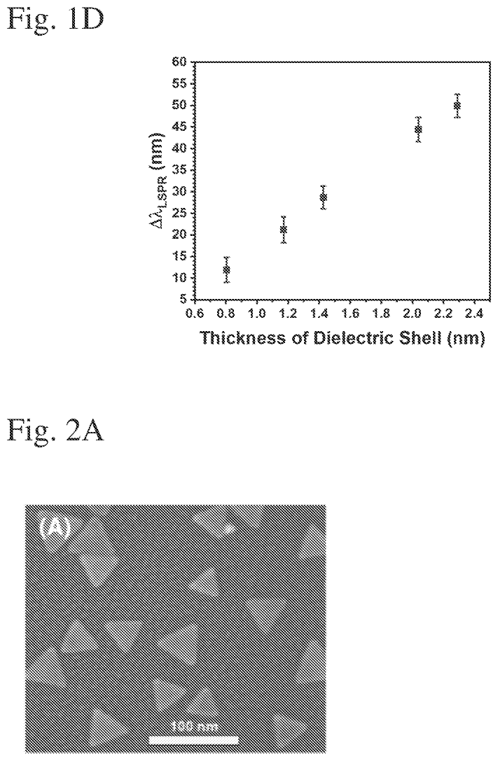

To overcome current challenges in bioanalytical assays and create more accurate, reliable and reproducible early disease diagnostics, we propose the fabrication and characterization of an entirely new, and highly adaptable nanoplasmonic biosensor (biosensing chip and assay switching protocol) capable of high sensitivity and simultaneous measurement of three important classes of biomarkers, i.e., microRNAs, long non-coding RNAs (lncRNAs), and proteins using a single UV visible spectrophotometer (multiplexing). Importantly, the biosensor uses an identical structural motif to detect these diverse biomolecules, programming detection by simply exchanging attached receptor molecules using light (FIG. 1A). This fabrication strategy yields a highly adaptable technique. The proposed biosensors utilize localized surface plasmon resonance (LSPR) properties of chemically-synthesized gold triangular nanoprisms (Au TNPs) coupled with the molecular dipole of zwitterionic surfaces to enhance plasmonic response and increase biosensor efficiency and sensitivity.

Plasmonic nanostructures provide an extraordinary range of structures, properties, and functions that can be exploited for a responsive and adoptive system if nanostructures can be effectively integrated with standard device technologies. As disclosed herein a new in vitro technology that has multiplexing biomolecular assay ability with high-throughput capability is provided. The unique aspects of the LSPR-based technology include: (1) an ultrasensitive and non-invasive optical-based assay capable of quantifying biomolecules in <10 μL human biofluid samples without any sample processing; (2) the ability to quantify both RNAs and proteins using identical workflow and device readout; (3) high selectivity and biocompatibility in a biosensing chip that can be constructed using low-cost plastics and scalable fabrication strategies to meet clinical demand.

This highly transformative approach, quantifying various biomarkers diagnostic for a disease state (e.g., a cancer patient sample), will build fundamental knowledge on disease diagnostics by connecting three important biological processes: transcription, epigenomics, and proteomics, that together will expedite a predictive and personalized approach to disease diagnosis and treatment.

As disclosed herein for the first time, applicant demonstrated a dramatic change in localized surface plasmon resonance (LSPR) properties of gold triangular nanoprisms (Au TNPs) functionalized with self-assembled monolayers (SAMs) of alkylthiols containing covalently bound spiropyran (SP) during their solid-state photoisomerization. An unprecedentedly large, ˜24 nm reversible shift (reproducible up to 5 cycles) of the TNP dipole peak (Δλ_{LSPR}) is observed during photo-isomerization between SP and merocyanine (MC) forms. Control experiments suggest that this shift is the combination of an increase in local dielectric environment surrounding the TNP (a 0.2 nm thickness change between SP and MC) and dipole-dipole interactions between TNP and the MC form. In one embodiment, starting with the MC form, applicant has designed and fabricated an LSPR-based, adaptable nanoplasmonic biosensor, the first of its kind, that is capable of detecting microRNAs (e.g., microRNA-10b, and -145) and proteins (e.g., nuclear mitotic apparatus protein-1, NuMA) from cancer patient biofluids (plasma and urine) at high attomolar concentration range with high specificity. Most importantly, these cancer biomarkers were detected with an identical biosensor construct by programmable exchange of DNA or protein receptor molecules that electrostatically adsorbed onto the activated MC form and detect either the microRNA or the protein.

The photo-isomerization of SP (our molecular switch) has been studied both for fundamental purposes, and potential applications in sensing, imaging and drug delivery. Light-induced isomerization of such a “molecular switch” in solid-state provides several investigative advantages: (1) fast response and no leaching, (2) non-destructive cycling and reduced photodegradation, (3) high precision of its structural conformation, and (4) on demand tuning and modulation of electronic properties and functions. Irradiation of SP by UV light (<400 nm) opens the oxygen-containing ring, leading to the formation of MC, whereas exposure to visible light (>450 nm) causes the reverse transition. In the literature, we find that the surface of inorganic nanostructures can be modified with SP-containing SAMs to modulate their LSPR via a self-assembly process in solution. Also, such solution phase functionalization of quantum dots influences their fluorescence properties due to alteration in energy transfer. Previously, chemically-tethered SP/MC attached to a solid surface was studied for fundamental purposes and biosensing application (Blonder et al, Journal of the American Chemical Society 1997, 119, 10467-10478). In this context, to the best of our knowledge, no report is available that demonstrates the optical response of metallic nanostructures (mainly LSPR properties) together with nanoplasmonic biosensor development utilizing SP-to-MC photoisomerization in solid-state.

Design of an appropriately constructed SP-SAM-modified plasmonic nanostructure that is a photoswitchable solid-state molecular device in which light induced changes in structural and electronic properties of the organic moiety may influence electronic properties, can be studied by monitoring LSPR properties. Such properties of metallic nanostructures depend not only on size, shape, and local dielectric environment but also short-range, inter-particle coupling. Therefore, an appropriate TNP-SAM-SP/MC construct in a well-defined physicochemical environment has the unique potential for development of advanced molecular electronics, highly efficient biosensors, and various other nanotechnology-based applications. We used a suite of spectroscopic techniques along with density functional theory calculations to investigate the photoisomerization of SP/MC covalently attached to Au TNPs through a SAM upon cycling exposure to UV and visible light. As disclosed herein short-range dipole-dipole (permanent and induced) interactions between a TNP and its zwitterionic MC surface substantially alter the LSPR properties and in addition result in an increase in optical response and high sensitivity in biomolecular assays. The LSPR properties have been applied to the detection of two different biological structures (protein and microRNA).

The accurate, reliable, fast, sensitive, selective, and reproducible detection of a variety of disease-related biomarkers are key in enabling point-of-care (POC) clinical diagnostics to increase patient survival. However, existing univariant, gold standard analytical techniques (RT-PCR, ELISA, etc.) generally operate with discrete formats that are unable to perform multiplexing assays and show inadequate sensitivity to low abundance biomarkers, specifically critical at disease onset, leading ultimately to false test results. Here, in accordance with one embodiment, a gold triangular nanoprism (Au TNP)-spiropyran (SP) zwitterionic structural motif was constructed, enabling multimodal, optical-based sample analyses (localized surface plasmon resonance (LSPR) and surface-enhanced Raman scattering (SERS)). Importantly, this motif is easily manipulated to detect different biomarker classes (RNA, protein) by simply changing the identity of the electrostatically adsorbed receptor molecules by light switching. No changes to biosensor structural motifs or workflow are required for this sensor adaptability. This strategy eliminates the need to develop new immobilization steps for different types of biomarkers, and therefore represents a transformative step towards simplifying advanced biosensor design, particularly in the context of POC clinical diagnostics.

Novel biosensor building blocks based on TNP-SP configuration, which can be easily and reversibly transformed into a zwitterionic structure (merocyanine, MC) through photochromatic isomerization, were utilized to investigate molecule-plasmon dipole interactions with the goal to achieve unprecedentedly high LSPR and SERS detection. The proposed TNP-MC structural motif represents a multifunctional and adaptable super-biosensor that through nanoscale engineering guided by fundamental scientific knowledge obtains the optimum TNP-MC interactions to generate highly sensitive biosensors with zeptomolar detection limit. The chemical fabrication approach builds biosensors from plastic multi-well plates, where each well can be treated as an independent biosensor. The biosensor has the capacity to assay a range of biomolecules, including microRNAs, lncRNAs, and proteins directly from biofluids of patients using a single 384 well plate and using a standard plate reader in absorption mode. Furthermore, the high SERS enhancement of these biosensors will provide strong Raman signals from specifically designed biomarker tags in order to fully mitigate false positive and false negative responses, the most critical concern for POC clinical diagnostics. Together, state-of-the art characterization methods, coupled with appropriate theoretical calculations, will make it possible to uncover unique structure-property-function relationships of great interest to scientific and engineering communities.

### Additional Embodiments

In accordance with embodiment 1, an adaptable nanoplasmonic biosensor for specific detection of target proteins and nucleic acids is provided, wherein said biosensor comprises
a localized surface plasmon resonance (LSPR) chip having an affixation surface and a functional surface;
a functionalized solid support; and
an LSPR antennae comprising a light inducible isomerizable compound, wherein
said affixation surface of the LSPR chip is covalently linked to said solid support, and said LSPR antennae is linked to said functional surface of the LSPR chip.

In accordance with embodiment 2, an adaptable nanoplasmonic biosensor of embodiment 1 is provided wherein the LSPR chip is a metal comprising triangular nanoprism, optionally wherein the metal is selected from the group consisting of gold, silver, copper, palladium, aluminum, or a combination thereof, and optionally wherein said LSPR chip is covalently linked to said solid support via a plurality of spacer molecules that comprise a poly-ethylene glycol moiety, an alkyl moiety, or a combination thereof.

In accordance with embodiment 3, an adaptable nanoplasmonic biosensor of embodiment 1 or 2 is provided wherein the LSPR chip is a gold triangular nanoprism (Au TNP) and said spacer molecules comprise a first end bound to the solid support and a second end comprising a functional group, optionally a thiol, that forms a covalently bond to group located on the affixation surface of the LSPR chip.

In accordance with embodiment 4, an adaptable nanoplasmonic biosensor of any one of embodiments 1-3 is provided wherein said LSPR antennae is linked to said functional surface of the LSPR chip via a plurality of spacer molecules that comprise a poly-ethylene glycol moiety, an alkyl moiety, or a combination thereof, optionally wherein the LSPR chip is a gold triangular nanoprism (Au TNP) and said spacer molecules comprise a first end bound to the LSPR antennae and a second end comprising a functional group, optionally a thiol, that forms a covalently bond to group located on the functional surface of the LSPR chip.

In accordance with embodiment 5, an adaptable nanoplasmonic biosensor of any one of embodiments 1-3 is provided wherein said LSPR antennae are covalently linked to said functional surface of the LSPR chip via a plurality of alkylthiolate spacer molecules, optionally wherein the alkylthiolate spacer molecules are nonanethiol or undecanethiol spacer molecules.

In accordance with embodiment 6, an adaptable nanoplasmonic biosensor of any one of embodiments 1-5 is provided wherein the LSPR chip is a gold triangular nanoprism (Au TNP) and said Au TNP has an average edge-length of between 30 and 50 nm.

In accordance with embodiment 7, an adaptable nanoplasmonic biosensor of any one of embodiments 1-6 is provided wherein the solid support is substantially transparent to electromagnetic radiation having a wavelength between 100 nm and 700 nm.

In accordance with embodiment 8, an adaptable nanoplasmonic biosensor of any one of embodiments 1-7 is provided wherein the light inducible isomerizable compound forms a zwitterion upon exposure to UV light and said light inducible isomerizable compounds are covalently linked to said functional surface of the LSPR chip via a plurality of alkylthiolate spacer molecules, optionally wherein the alkylthiolate spacer molecules are undecanethiol spacer molecules.

In accordance with embodiment 9, an adaptable nanoplasmonic biosensor of any one of embodiments 1-8 is provided wherein the light inducible isomerizable compound has the general structure of

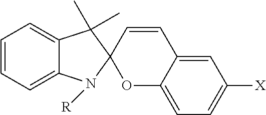

wherein R is selected from the group consisting of —(CH₂)ₙN⁺(CH₃)₃, wherein n is an integer selected from 1 to 6, and
X is —NO₂, —CONH₂ or —COOH.

In accordance with embodiment 10, an adaptable nanoplasmonic biosensor of any one of embodiments 1-9 is provided wherein the light inducible isomerizable compound has the structure of

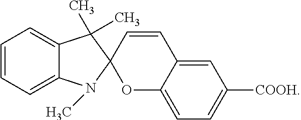 [SMILES: CN1c2ccccc2C(C)(C)C12C=Cc1cc(C(=O)O)ccc1O2]

In accordance with embodiment 11, an adaptable nanoplasmonic biosensor of any one of embodiments 1-10 is provided wherein the LSPR antennae comprises the structure:

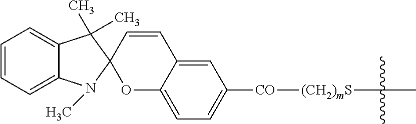

wherein m is an integer selected from 9 to 13.

In accordance with embodiment 12, an adaptable nanoplasmonic biosensor of embodiment 11 is provided wherein m is 11 (SP-UT).

In accordance with embodiment 13, an adaptable nanoplasmonic biosensor of any one of embodiments 1-12 is provided wherein the biosensor further comprises a plurality of undecanethiol (UT) polymers linked to said functional surface of the LSPR chip.

In accordance with embodiment 14, an adaptable nanoplasmonic biosensor of embodiment 13 is provided wherein the LSPR chip comprises a 75%:25% mixture of SP-UT and UT linked to said functional surface of the LSPR chip.

In accordance with embodiment 15, an adaptable nanoplasmonic biosensor of any one of embodiments 1-14 is provided wherein the Au TNP has an average edge-length of between 30 and 50 nm, optionally having an average edge-length of about 42 nm and 8 nm height (AFM analysis) that is attached onto thiolated silane-functionalized solid support that is substantially transparent to electromagnetic radiation having a wavelength between 100 nm and 700 nm.

In accordance with embodiment 16, a method of detecting the presence of a first and second analyte in a biological sample through the use of a single device utilizing an identical signal output for the detection of both the first and second analyte is provided. The method comprises the steps of
a) providing a nanoplasmonic biosensor according to any one of embodiments 1-15;
b) exposing the biosensor to UV light to induce photoisomerization of said isomerizable compound and produce the zwitterion form of said compound;
c) contacting the zwitterion form of said compound with a first ligand that specifically binds to said first analyte, wherein the first ligand electrostatically binds to the zwitterion form of said compound to form a first ligand complex;
d) contacting said first ligand complex with a biological sample;
e) conducting LSPR analysis of said nanoplasmonic biosensor after step d);
f) optionally rinsing said first ligand complex with a buffer to promote charge screening to disrupt binding of the first ligand to the zwitterion form of said compound;
g) exposing the biosensor to visible light to regenerate the non-zwitterion form of said compound and rinsing said biosensor to remove unbound material;
h) exposing the biosensor to UV light to induce photoisomerization of said isomerizable compound and produce the zwitterion form of said compound;
i) contacting the zwitterion form of said compound with a second ligand that specifically binds to said second analyte, wherein said second analyte electrostatically binds to the zwitterion form of said compound to form a second ligand complex;
j) contacting said second ligand complex with a biological sample;
k) conducting LSPR analysis of said nanoplasmonic biosensor after step j).

In accordance with embodiment 17, a method of embodiment 16 is provided wherein said non-zwitterion form of said compound comprises the structure of
of

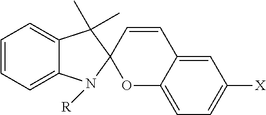

wherein R is selected from the group consisting of —(CH₂)ₙN⁺(CH₃)₃, wherein n is an integer selected from 1 to 6, and
X is —NO₂, —CONH₂ or —COOH; and
   said zwitterion form of said compound comprises the structure of

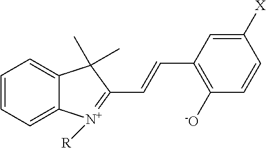

wherein R is selected from the group consisting of —(CH₂)ₙN⁺(CH₃)₃, wherein n is an integer selected from 1 to 6, and
X is —NO₂, —CONH₂ or —COOH.

In accordance with embodiment 18, a method of embodiment 16 or 17 is provided wherein the LSPR chip is a gold triangular nanoprism (Au TNP), said light inducible isomerizable compounds are covalently linked to said functional surface of said Au TNP via a plurality of undecanethiol spacer molecules, wherein said functional surface of said Au TNP further comprises a plurality of undecanethiol (UT) polymers linked to said functional surface of the LSPR chip.

In accordance with embodiment 19, a method of any one of embodiments 16-18 is provided wherein the steps of conducting LSPR analysis comprises measuring an absorption spectrum of the LSPR antenna, the absorption spectrum having a peak wavelength; and determining the presence or quantity of the first and second analyte in said sample based on the peak wavelength.

In accordance with embodiment 20, a method of any one of embodiments 16-19 is provided wherein the method of
detecting the presence of the first analyte in said sample comprises measuring a first absorption spectrum of the LSPR antenna after step c) and measuring a second absorption spectrum of the LSPR antenna after step d) and determining the difference between the peak wavelength of the first and second measured absorption spectrum of the LSPR antenna; and
detecting the presence of the second analyte in said sample comprises measuring a third absorption spectrum of the LSPR antenna after step i) and measuring a fourth absorption spectrum of the LSPR antenna after step j) and determining the difference between the peak wavelength of the third and fourth measured absorption spectrum of the LSPR antenna.

In accordance with embodiment 21, a method of any one of embodiments 16-20 is provided wherein the first analyte is a protein, the second analyte is a nucleic acid, the first ligand is a protein receptor that specifically binds to said protein and the second ligand is a nucleic acid sequence that specifically binds to said second analyte nucleic acid.

In accordance with embodiment 22, a method of any one of embodiments 16-21 is provided wherein the first analyte is a first RNA, the second analyte is a second RNA, the first ligand is a first nucleic acid sequence that specifically binds to said first RNA and the second ligand is a nucleic acid sequence that specifically binds to said second RNA, wherein the first and second RNAs have difference nucleic acid sequences.

### EXAMPLE 1

Fabrication of nanoplasmonic biosensor chip.

In one embodiment, fabrication of nanoplasmonic biosensor chip and detection of microRNAs and proteins were performed as follows:
(FIG. 1A) Au TNPs with 42 nm edge length and 8 nm height (AFM analysis) were attached onto thiolated silane-functionalized glass coverslips by 30 min incubation. The TNP surface was then modified with a 75:25 mole ratio of SP-nonanethiol and nonanethiol SAMs in ethanol and then the coverslip was exposed to UV light (365 nm, 10 min) to generate MC-nonanethiol structures. The coverslips were incubated in PBS buffer containing either a ssDNA or a receptor protein.
(FIG. 1B) -ssDNAs or (FIG. 1C) protein electrostatically adsorbed onto the charged, MC surface.
(FIG. 1D) The LSPR biosensor peak was measured in PBS buffer. The biosensors were incubated overnight in human plasma supplemented with microRNA or protein. Here, -ssDNA (receptor) has the complementary base pair for the target microRNA (analyte) (FIG. 1E), and the receptor protein specifically binds to analyte protein (FIG. 1F). The analyte-bound chips were thoroughly washed with PBS buffer and placed in a cuvette containing PBS buffer, and then LSPR was measured using a traditional UV-visible spectroscopy. The peak shift magnitude (DI) is dictated by the amount of biosensor-bound analyte. Plot of DI vs log of analyte concentration was used to determine LOD. A standard dimension of a biosensor chip in accordance with the present invention is about 2.5 by about 0.6 cm.

### Materials and Methods

Synthesis of SP-COO—(CH₂)₁₁—SH. The synthesis of thiol spiropyran, SP-COO—(CH₂)₁₁—SH, was conducted as follows: The synthesis consisted of three major steps: synthesis of 3-formyl-4-hydroxybenzoic acid (BA-COOH), synthesis of spiropyran carboxylic acid (SP-COOH), and the synthesis of spiropyran thiol (SP-COO—(CH₂)₁₁—SH). Briefly, BA-COOH was synthesized utilizing a Duff reaction with 5 g (36.2 mmol) of 4-hydroxy-benzoic acid (BA), which was added to a 100 mL 2-neck round bottom flask and mixed with 15 mL of trifluoroacetic acid (TFA) under nitrogen with stirring for 30 minutes at room temperature. Separately, 5 g (36.2 mmol) of hexamethylenetetramine (HMTA) was mixed with 15 mL of TFA. The mixture was then added dropwise to the round bottom flask containing the BA. After completion of the addition, the reaction vessel was transferred to a 250° C. preheated oil bath and refluxed for 2-3 hours under nitrogen. ESI analysis was conducted to confirm BA was fully reacted by the disappearance of m/z 138. BA-COOH was then precipitated with 4 N hydrochloric acid (HCl) over 3 hours and recovered using vacuum filtration. The sample was dried under vacuum overnight. ESI-MS analysis was used to confirm the presence of BA-COOH, MS (ESI): m/z=166.

The second step of the process facilitated the synthesis of SP-COOH. 2.5 g (15.1 mmol) BA-COOH was added to a 100 mL 2-neck round bottom flask under nitrogen. 40 mL of purged ethanol was added. 3.8 mL (21.8 mmol) of 1,3,3-trimethyl-2-methylene indoline (TMMI) was obtained and added under nitrogen, with stirring. The reaction mixture was then placed in an oil bath at 175° C. and refluxed for approximately 2 hours. An ESI-MS analysis was then conducted to determine that BA-COOH was no longer present by disappearance of m/z 66. SP-COOH was purified using a silica gel column with a solvent gradient of dichloromethane (DCM) and methanol (MeOH). The SP-COOH product was obtained at 5% MeOH and the remainder with 10% MeOH. The fractions were concentrated using a rotovap and then dried under vacuum overnight. An ESI-MS analysis of the product was conducted to confirm presence of SP-COOH product, MS (ESI): m/z=320.

The final step of the process utilizes a DCC-NHS coupling of SP-COOH and 11-mercaptoundecanol to produce the final product SP-COO—(CH₂)₁₁—SH. 4.2 g (13.2 mmol) of SP-COOH was added to 100 mL of purged dichloromethane (DCM) in a 250 mL 2-neck round bottom flask under nitrogen. 3.2 g (15.9 mmol) of 11-mercaptoundecanol and 0.18 g (1.5 mmol) of dimethyl amino pyridine (DMAP) were added individually directly to the reaction mixture. The reaction was stirred under nitrogen in an ice bath until the internal temperature reached 0° C. Separately, 3.3 g (13.2 mmol) of N,N-dicyclohexylcarbdiimide (DCC) was dissolved in 100 mL of purged DCM. The DCC solution was added to the reaction flask dropwise over 40 minutes. The reaction then slowly reached room temperature with stirring and was allowed to react overnight under nitrogen. Before the reaction was stopped, an ESI analysis was conducted to determine that SP-COOH was no longer present by the disappearance of m/z 320. The reaction solution was then concentrated under rotovap and purified with a silica gel column. The column used a hexane (HEX) and ethyl acetate (EtOAc) gradient to purify and obtained the desired SP-COO—(CH₂)₁₁—SH product. The product was obtained at 10% EtOAc. The fractions were concentrated using a rotovap and then dried under vacuum overnight. An ESI-MS analysis of the product was conducted to confirm presence of SP-COO—(CH₂)₁₁—SH by the appearance of molecular weight 506 g/mol, MS (ESI): m/z=508 [M]⁺.

### Development of Biomarker Calibration Plots in Human Plasma and Urine

The LSPR absorption spectra of the MC activated Zwitterionic biosensors were collected in air to determine λ_{LSPR}. The biosensors were incubated overnight in 6 mL of 10 μM -ssDNA-145, -ssDNA-10b or 100 ng/ml Anti-NuMA. The next day, coverslips were dried and λ_{LSPR} was determined in air. The functionalized biosensors were then incubated overnight in 6 mL of microRNA-145, microRNA-10b or NuMA at different concentrations (ranging from 10 nM to 100 aM) in 10% human plasma/PBS buffer solution for -ssDNA or 10% human urine/RNase-free water solution for NuMA. Receptor-bound biosensors were washed with PBS buffer for -ssDNA or RNase free water for NuMA to remove any nonspecifically adsorbed biomolecules, dried under N₂ flow, and the final λ_{LSPR} was determined. False positive analysis was conducted by incubating the biosensors in a PBS buffer and RNase-free water solutions containing receptor but no analyte. False negative analysis was conducted by incubating the biosensors without the receptor in 10 nM biomarker solution.

### Spectroscopy and Microscopy Characterizations

Absorption spectra in the range of 300-1100 nm were collected with a Varian Cary 50 Scan UV-visible spectrophotometer using 1 cm quartz cuvettes. All absorption spectra were collected in air with glass coverslips that had been dried under N₂ flow. A blank glass coverslip was used as a background and an Au TNP-functionalized coverslip incubated in PBS buffer or human biofluid was considered the reference (blank). The chemically synthesized Au TNPs attached onto the silanized glass coverslips were characterized using scanning electron microscopy (SEM) and atomic force microscopy (AFM). SERS analysis was performed using a Foster and Freman Foram 785 HP Raman system with a 785 nm diode laser excitation source with 20 mW of power and 5-μm spot size. The SERS data were acquired for plasmonic patches with 10 scans at 20 mW power, from 400-2000 cm⁻¹ and a 16 see acquisition time. Automatic baseline correction was performed using OMNIC software before acquired spectra were plotted. All SERS measurements were plotted, and the average Raman intensity was obtained, using Origin software.

### Silanization of Glass Coverslips

Glass coverslips with 25×25 mm dimension were functionalized according to our previously published procedure (Joshi et al, J. Phys. Chem. C 2012, 116, 20990-21000). Briefly, glass coverslips were incubated in a 10% (v/v) aqueous RBS 35 detergent solution heated to 90° C. and were sonicated for 15 minutes. Nanopure water was then used to rinse the coverslips thoroughly, and then the coverslips were incubated in a 1:1 (v/v) hydrochloric acid:methanol solution for 30 minutes. Following additional rinsing with nanopure water, coverslips were dried overnight in a vacuum oven at 60° C. The following day, the coverslips were brought to room temperature, and then incubated in a 15% (v/v) solution of MPTMS in N₂ purged ethanol for 30 min. The coverslips were then covered with EtOH and sonicated 3-5 times for 10 min each. Finally, coverslips were dried in a vacuum oven at 120° C. for a minimum of 3 hr. The prepared MPTMS functionalized coverslips were then stored at 4° C. for a maximum of one week.

### Synthesis of Gold Triangular Nanoprisms (Au TNPs).

Au TNPs were chemically synthesized according to our previously developed procedure with minor modification (Liyanage et al, Nano Lett. 2020, 20, 192-200). Briefly, Et₃PAu(I)CI (18.0 mg, 0.05 mmol) was dissolved in 40 mL of N₂ purged CH₃CN and the solution was stirred for 10 min at room temperature. Stirring was stopped to facilitate the rapid addition of 0.038 mL (0.273 mmol) of TEA into the gold salt solution and then the mixture was heated with resumed stirring. When the temperature of the reaction mixture reached 38° C., 0.6 mL of PMHS was added to form a undisturbed bubble on the side of the reaction flask and then the reaction was allowed to proceeded with slow stirring while the temperature was maintained between 38-41° C. During this time, the color of the solution changed from colorless to pink, purple, light blue and then finally a dark navy blue. This dark navy-blue color indicates that the Au TNPs have formed and are approaching the correct edge-length and thickness. At this point, the solution was checked by localized surface plasmon resonance (LSPR) to confirm a stable dipole peak (λ_{LSPR}) position at 800 nm in CH₃CN. If present, the reaction was stopped by removing it from the hot plate followed by centrifugation at 7000 rpm for 10 seconds. Finally, previously prepared MPTMS-functionalized coverslips were incubated in the freshly prepared Au TNPs solution for 1 hr, rinsed with acetonitrile, dried under N₂ flow, and then stored under nitrogen at 4° C.

### Dielectric Shell Thickness (Thiol Chain Length) Study

In order to determine the contribution chain length has on λ_{LSPR}, five thiols with increasing number of CH₂ units were selected. 1-hexanethiol, 1-nonanethiol, 1-undecanethiol, 1-hexanedecanethiol and 1-octadecanethiol were chosen and prepared in 1.0mM solutions in N₂ purged EtOH, incubated in silanized glass coverslip bound Au TNPs for overnight. The next day, the coverslips were rinsed with EtOH, dried under N₂ flow and the λ_{LSPR} was determined.

### Data Processing and Statistical Analysis

The λ_{LSPR} was obtained through curve fitting using Origin software according to the maxima of the UV-visible absorption spectra, and the change in the LSPR dipole peak (Δλ_{LSPR}) was determined by taking the difference between the λ_{LSPR} of the biosensors before and after attachment of the biomarker. Calibration curves were obtained by plotting Δλ_{LSPR} vs. biomarker concentration, with concentration being plotted on the axis in log scale in order to investigate non-specific adsorption at a lower concentration range. The calibration curve equation was determined through linear regression on Origin. Finally, the LOD was derived by using a Z value of the blank (Z=mean+3σ, σ=standard deviation of blank), which was obtained from six Δλ_{LSPR} measurements using six different biosensors and substituting the Z value into the “Y” in the calibration curve equation allowing for the LOD concentration (“X”) to be obtained. Concentration of target antibodies in patient samples were determined from the calibration curves developed in plasma or urine, with Δλ_{LSPR} value and corresponding concentrations obtained from the average of six measurements. Each sample was independently analyzed twice (two weeks apart). Paired two-tailed t-test and area under the curve (AUC) of the receiver operating characteristic (ROC) graphs were plotted using GraphPad Prism. Paired two-tailed t-test used the following p value style: 0.1234 (ns), 0.0332 (*), 0.0021 (**), 0.0002 (***), <0.0001 (****), and were performed at the 95% confidence interval. The AUC of ROC was also performed at the 95% confidence interval.

### Time-Dependent Density Functional Theory (TDDFT) Calculations

TDDFT calculations were performed to determine dipole moment and Raman vibrational frequencies of SP-UT and MC-UT. Calculations were performed using Gaussian16 with B3LYP hybrid exchange correlation functional and 6-311+G ** basis set for SP-UT and MC-UT. To visualize the optimized geometry of each molecules, as well as to adding vibraitonal modes, Gaussview 6.0.16 was used.

### Explanation of Surface Plasmon Dipole and Molecular Dipole Interactions

A simple description of induced polarization and dipolar contribution is given by the classical electrodynamic interaction, as described by Eq. 1.

$$\frac{κ - 1}{κ + 2} = \frac{N}{3 {ε}_{0}} ( {α}_{0} + \frac{{μ}^{2}}{3 kT} ) \qquad ( 1 )$$

Here, κ is the dielectric constant, ε₀ is the permittivity of free space, N is the number of molecules, α₀ is the molecular polarizability (induced dipole moment), μ is the permanent dipole moment, k is the Boltzmann constant, and T is the temperature. For simplicity, molar mass and density of surface functionalized molecules were avoided. In the absence of high magnetic polarizability at visible frequency for Au, κ=n², where n is the index of refraction, leading to Lorentz and Lorenz relation (Eq. 2).

$${n}^{2} = \frac{9 {ε}_{0} KT + 2 N ( 3 {α}_{0} KT + {μ}^{2} )}{9 {ε}_{0} KT - N ( 3 {α}_{0} KT + {μ}^{2} )} \qquad ( 2 )$$

Therefore, an increase in permanent dipole moment would increase the overall n value at a constant temperature. Taken together, the MC form with a 23.78 D dipole moment would change the n value significantly more in comparison to SP with 2.77 D. This nanostructure-molecule dipole-dipole interaction can be compared to near-field dipole-dipole interactions between two adjacent nanostructures that result in a LSPR red-shift. Electrodynamic coupling is a multivariable interaction, which can be influenced by the induced dipole moment of the organic ligand shell and the spatial organization of both TNPs and their surface ligands. Herein, we considered only the simplest and most important permanent dipole-dipole interactions.

### EXAMPLE 2

### Photoswitchable Machine-Engineered Plasmonic Nanosystem

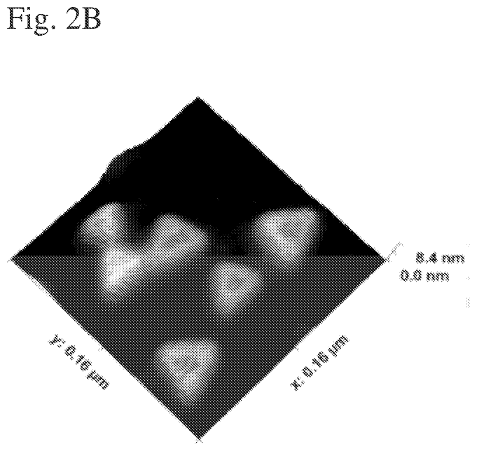

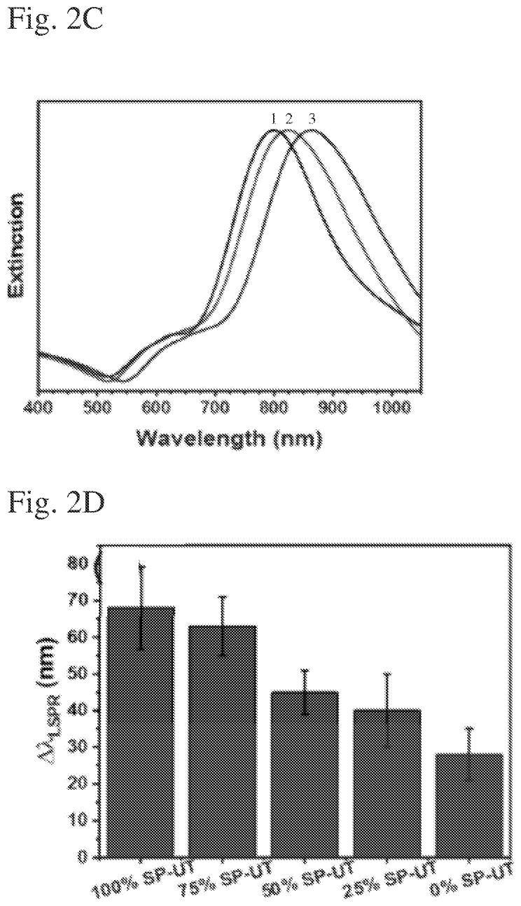

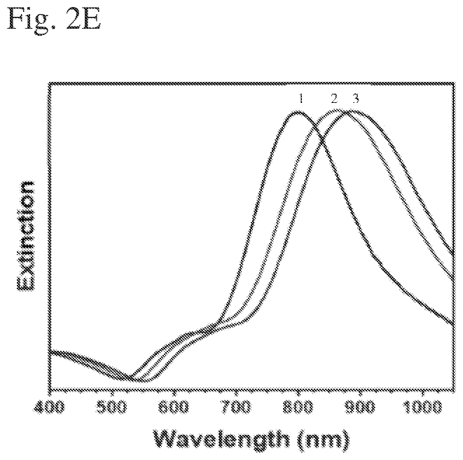

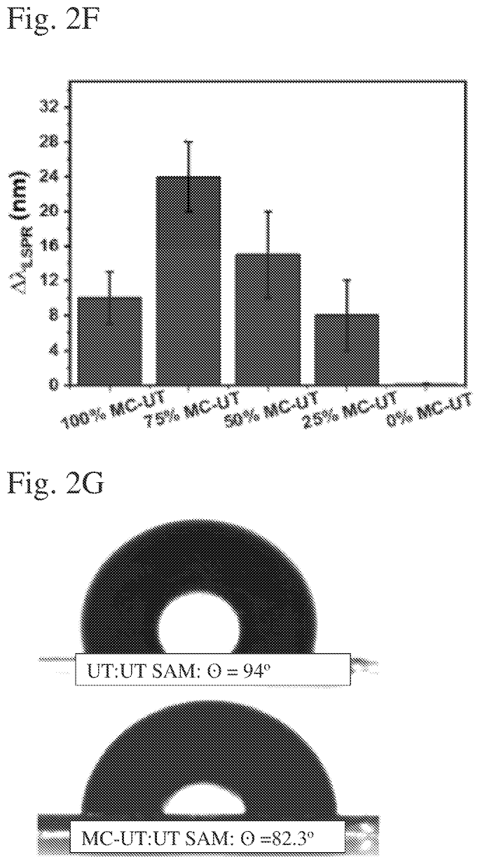

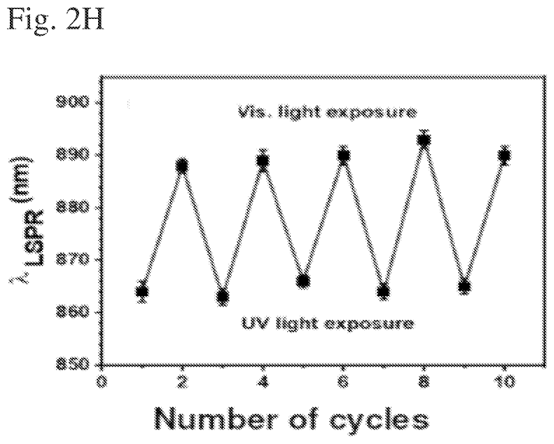

To construct a plasmonic-based molecular device consisting molecular switches (machines), we functionalized Au TNPs attached to a silanized glass coverslip with an SP-linked undecanethiol (SP-UT) SAM, with the result shown schematically in FIG. 1A. We selected Au TNPs due to their excellent photostability and high LSPR response that originates from high electromagnetic (EM) field enhancement at the sharp tips and corners. Au TNPs have been established to one type of nanostructures suitable for highly sensitive molecular and biological sensor construction. We started with triethylamine (TEA)-passivated TNPs with an average 42 nm edge length and ˜8 nm height (see FIGS. 2A and 2B) that display λ_{LSPR} at 800 nm in air (see FIG. 2C). Ligand exchange of these TNPs with 100% SP-UT results in a Δλ_{LSPR} of 68±11 nm in air. This large shift could potentially result exclusively from the change in the thickness of local dielectric shell originating from the hydrocarbon SAM around the TNPs versus TEA. In contrast, a much smaller Δλ_{LSPR} of 28±7 nm is observed when TNPs were functionalized with only undecanethiol (UT, without SP) SAMs. We modeled (ChemDraw 3D) a fully extended UT and SP-UT and found theoretical thicknesses of 1.43 and 2.66 nm, respectively. The difference in thickness between UT and SP-UT SAM is ˜1.23 nm and provides an increase in wavelength shift of 40 nm, which is unexpectedly high. To further validate our theoretical argument of the importance of more than just dielectric constant, we prepared TNPs with SAMs of different length alkylthiols by varying methylene unit (—CH₂) number to determine the Δλ_{LSPR} as a function of thickness of the local dielectric shell. These measurements predict that a 2.66 nm thick pure alkanethiol dielectric shell would produce Δλ_{LSPR} of 55 nm, which is significantly smaller than our experimentally determined value of 68 nm. As shown in FIG. 2D, the Δλ_{LSPR} value decreases monotonically with a lower contribution of SP-UT to the SAMs by varying the mole percentage of SP-UT and UT, suggesting an increasing contribution to wavelength shift from the structurally complex and longer SP-UT.

One of the main virtues of molecular machines is that they undergo structural isomerization under the influence of external stimuli. Next we investigated exposure of the SP conformation to UV light producing MC and how this effects the bound TNPs. This photoisomerization should influence the molecule-nanostructure interfacial interactions and thus the LSPR property of the adjacent nanostructure. Importantly, observation of light-induced photo-isomerization of organic molecules chemically tethered onto nanostructures by monitoring their LSPR properties in solid-state is extremely rare. Several mixed SAM (varying the mole percentage of SP-UT and UT)-functionalized Au TNPs were exposed to UV light for 10 min in solid-state and the λ_{LSPR} position was measured. As illustrated in FIG. 2E, conversion of SP to MC structure results in a red-shift of λ_{LSPR} position. Interestingly, the interaction is not monotonic in percent of SP-UT versus UT, and the highest value (Δλ_{LSPR}=24±4 nm) is observed for a 75%:25% mixture of SP-UT and UT SAMs (FIG. 2F). In contrast, 100% SP-UT SAMs provide Δλ_{LSPR} of 10 nm. We do not fully understand this different LSPR response to mixed SAM composition but believe that the steric hindrance in the solid state with a 100% SP-UT SAM does not allow all SP molecules to convert to the MC form during light exposure, whereas a lower percentage of SP-UT SAMs would produce a relatively smaller change in the dielectric shell thickness. It has been reported that SP chemically-tethered onto a solid-substrate requires appropriate assembly control to reduce the steric hindrance in order to observe photo-isomerization to MC. In our system, equal chain length of alkylthiol linker and spacer (11 methylene units) is expected to produce a dense lower layer SAM, whereas the 75% SP-UT mole ratio should allow geometric freedom for SP to undergo structural isomerization above the lower layer SAM under light irradiation.

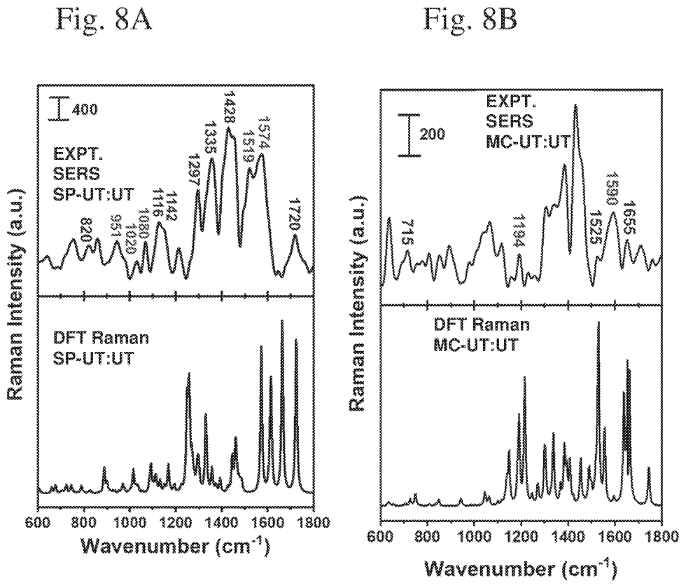

To further confirm that conversion to the MC form has caused the observed LSPR peak red shift, we characterized both SP-and MC-modified TNPs in solid-state by surface-enhanced Raman scattering (SERS) and compared the experimental spectra with DFT-calculated Raman spectra. The experimental Raman stretches of closed-ring SP for various vibrations are in agreement with the literature and/or calculated spectra. Importantly, new C═N stretch and C—N—C scissoring caused by the ring opening of SP to MC appear at 1525 and 715 cm⁻¹, respectively. Additionally, ring opening also causes loss of the O—C—N stretch at 951 cm⁻¹ in the SERS spectrum of MC (see Table 1, Table 2 and FIG. 8). SP is a closed-ring aromatic structure with low polarity whereas MC is an open zwitterionic structure with high polarity, which can be verified by conducting a water contact angle measurement. As shown in FIG. 2G, 75:25 mixed SP-UT and UT SAMs has a contact angle of 94.0°, suggesting hydrophobicity. On the other hand, formation of hydroxyl and quaternary ammonium sites (see FIG. 1A) by light exposure in the mixed MC-UT and UT SAMs causes a contact angle of 82.3°, which suggests a hydrophilic surface. The trend in water contact angle values for our system is in agreement with the literature where SP-containing molecules were tethered on a glass surface. Taken together, our SERS and water contact angle measurements unequivocally prove that UV light exposure converted SP to MC that results in a λ_{LSPR} red-shift of ˜24 nm.

TABLE 1
Comparison of DFT-calculated Raman vibration stretches of
SP-UT and experimentally obtained SERS spectra of SP-UT:UT
SAM-functionalized Au TNPs.

| | Spiropyran | | Experimental |
|---|---|---|---|
| Vibrational Descriptions | Mode | DFT | SERS/cm⁻¹ |
| v (C═O) Carboxylic Group | 184 | 1723 | 1720 |
| δᵣ (C—H) (CH_{2;} Alkyl Chain) | 156 | 1422 | 1428 |
| v (C—N) Ring B | 140 | 1350 | 1355 |
| v (C—C) Ring B | 132 | 1295 | 1297 |
| y (N—CH₃) Ring B | 115 | 1142 | 1142 |
| y (CH₂) Alkyl chain | 111 | 1121 | 1116 |
| v (C—O) Ring C | 77 | 818 | 820 |

v = stretching, δ = in plane deformation (sc = scissoring, r = rocking), γ = out of plane deformation (w = wagging, t = twisting), β = breathing
A = Ring A; B = Ring B; C = Ring C

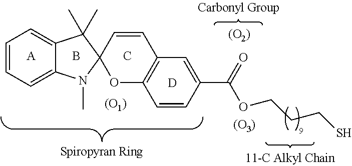

TABLE 2
Comparison of DFT-calculated Raman vibration stretches of MC-UT and
experimentally obtained SERS spectra of MC-UT:UT-SAM
functionalized Au TNPs.

| | Merocyanine | | Experimental |
|---|---|---|---|
| Vibrational Descriptions | Mode | DFT | SERS/cm⁻¹ |
| v (C═C) Trans Alkene | 182 | 1655 | 1655 |
| v (C═N) Ring B | 178 | 1530 | 1525 |
| δ_{sc} (C—N—C) Ring B | 65 | 720 | 715 |

v = stretching, δ = in plane deformation (sc = scissoring, r = rocking), γ = out of plane deformation (w = wagging, t = twisting), β = breathing
A = Ring A; B = Ring B; C = Ring C

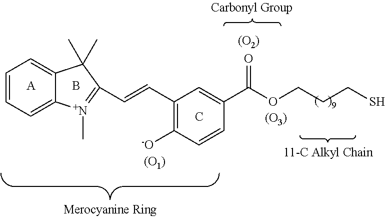

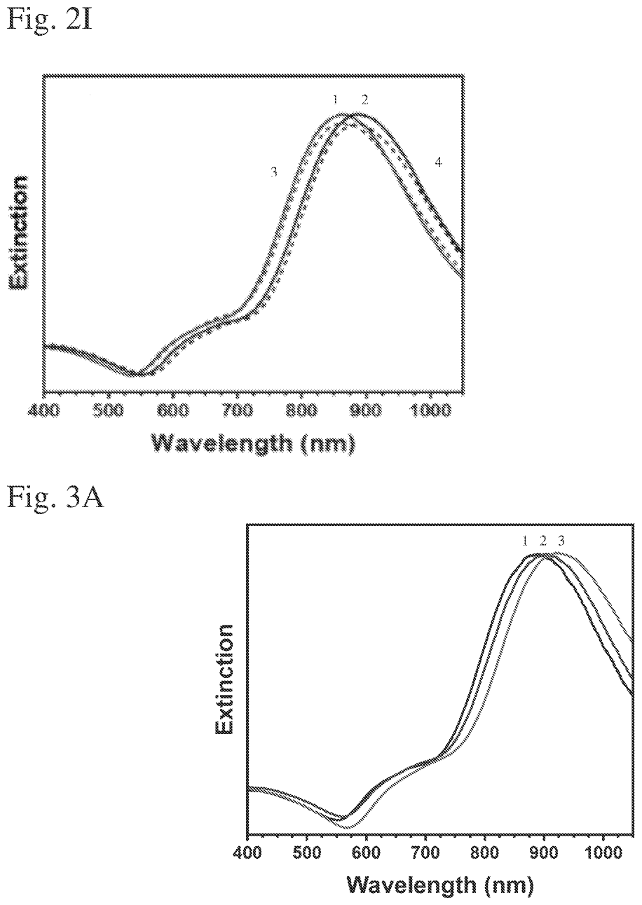

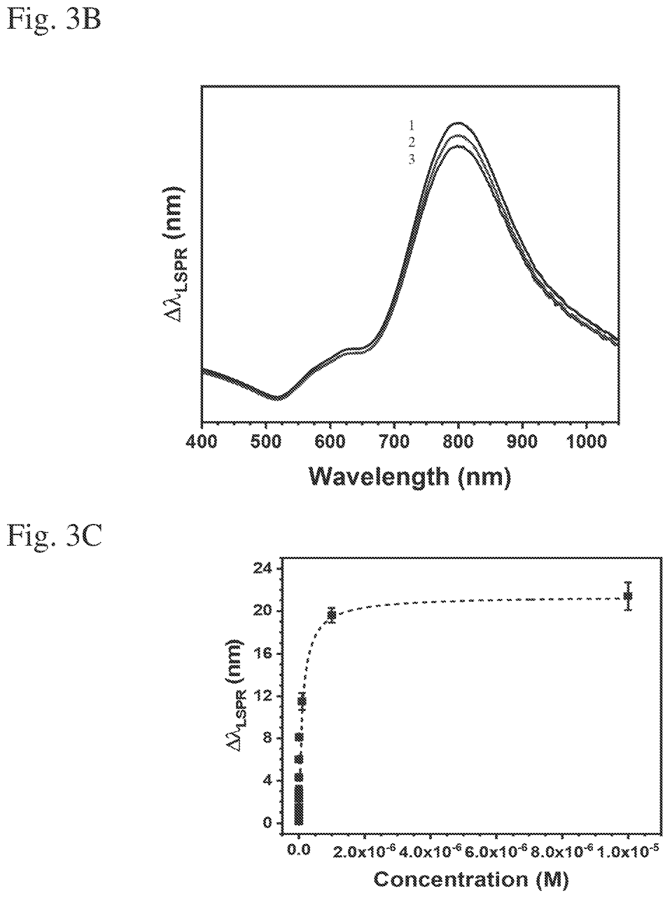

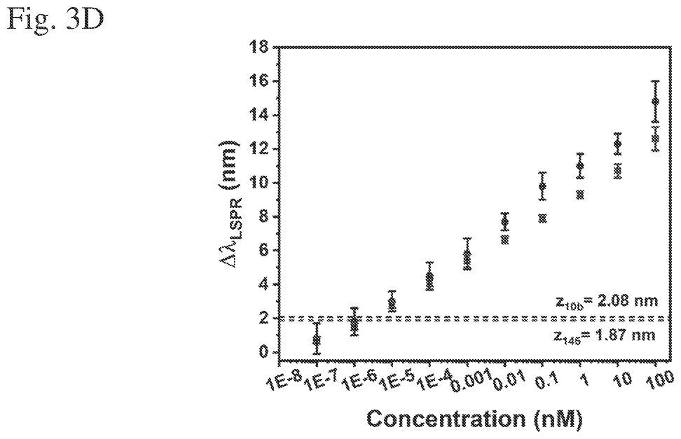

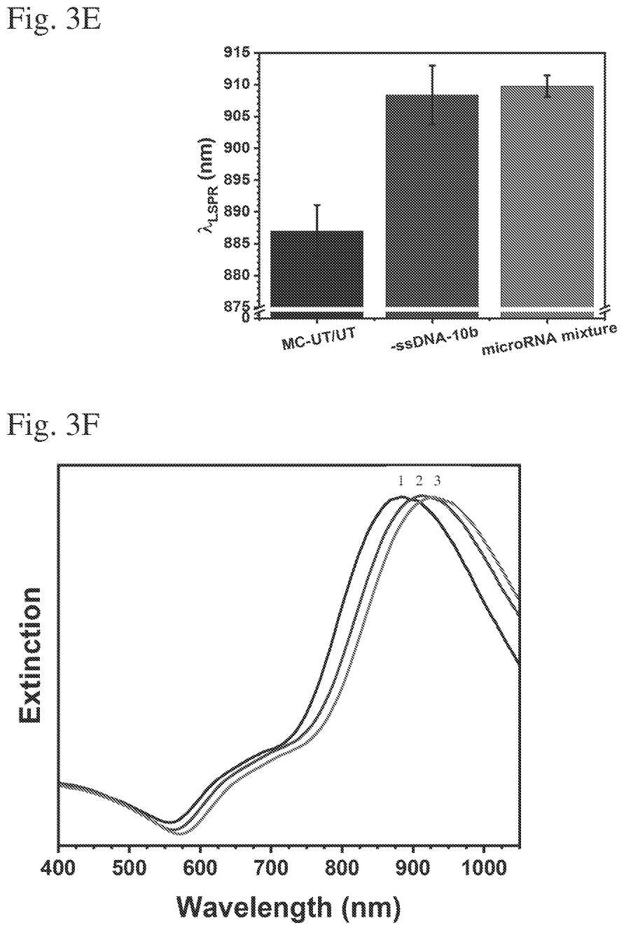

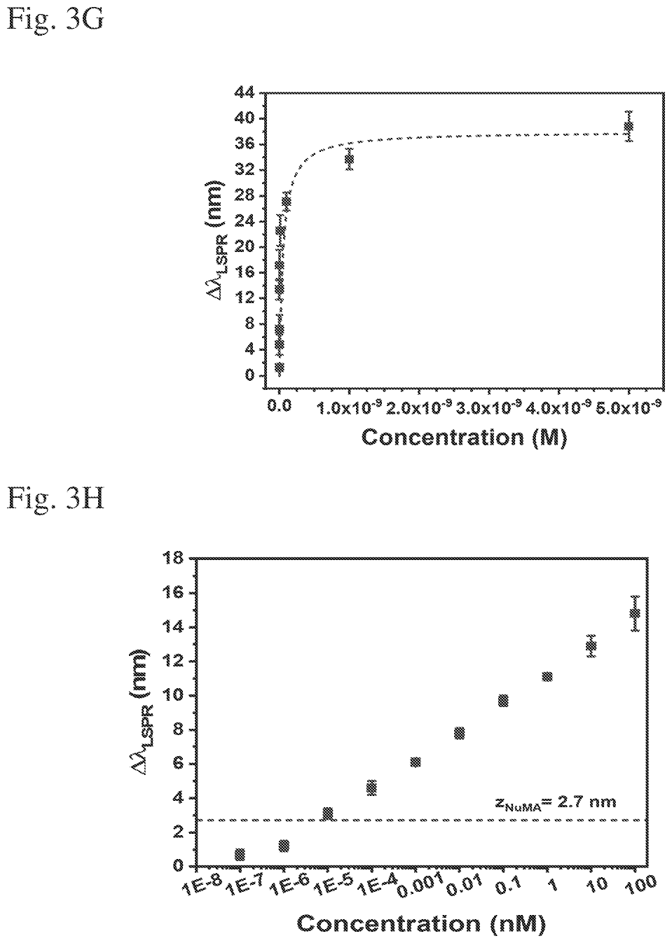

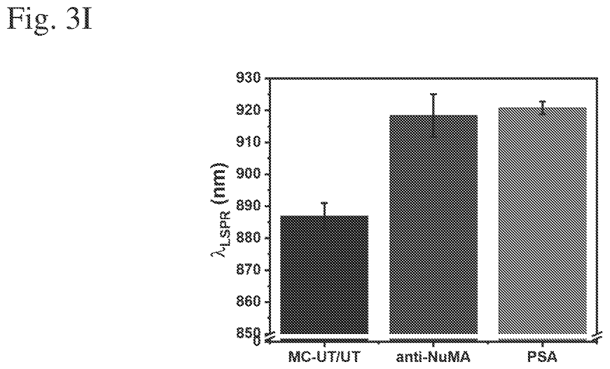

Achieving reversible photoswitching between SP and MC conformations is an important component in fabricating an adaptable, LSPR-based biosensing device. Reversible conformational changes of such “molecular machines” under repeated light exposure require an appropriate geometric construct at the nanoscale level. Additionally, plasmonic nanostructures and their surface coating, e.g., SAMs, should be stable over a long period of time. FIG. 2H represents the Δλ_{LSPR} upon repeated cycling of our photo-switch-based molecular machine between UV and Vis light exposures. Even after five full cycles of SP-MC conformational changes, the nanosystem displays a nearly identical Δλ_{LSPR} shift of an average 24 nm. This result indicates that our system is stable and can undergo reversible switching without compromising its efficiency. We believe that an appropriate packing of the SAM could be an important factor in allowing the reversible photoswitching over several cycles. Moreover, the entire photo-isomerization study was conducted in air, therefore there is a negligible chance for the zwitterion MC form to be involved in a chemical side reaction such as proton capture, which could prevent the transformation of the MC to SP form. FIG. 2I shows the extinction spectra after the first exposure cycle and then after the fifth cycle, where no dramatic change in the λ_{LSPR} value and extinction peak shape are observed. This consistency further suggests that during cycling light exposure, the Au—S bond remains intact. This is because the soft donor-soft acceptor interaction between S and Au provides high chemical stability. Such stability is important for our next stage, which is the design of a plasmonic-based adaptable biosensor with light-controlled receptor binding properties in solution. It has been shown that light exposure to metallic nanoparticles can result in photooxidation and charge accumulation, which influences their LSPR properties. To verify that the observed, reversible 24 nm shift of our nanosystem upon exposure to UV and visible light is due to photo-reversibility of SP and MC forms and not due to photooxidation and charge accumulation, we exposed TEA-passivated Au TNPs to UV light for 10 min and then collected the extinction spectra. No apparent change in the λ_{LSPR} position and shape of the extinction spectrum were observed (FIG. 3B). Similarly, visible light exposure does not show any change in the LSPR properties as well. This control experiment indicates that the observed reversible shifts in the λ_{LSPR} position are solely due to reversible photoisomerization between SP and MC structures.

Photo-isomerization between SP and MC of our 75:25 SP-UT and UT mixed SAM-functionalized Au TNP provides Δλ_{LSPR} value of 24 nm. Photoswitching of SP to MC causes an increase in the height of the SAM. Based on the extended geometry of SP-UT and MC-UT, we calculated the molecular lengths between the Au atom to the furthest atom in the aromatic ring to be 2.66 and 2.92 nm, respectively (FIGS. 1B and 1C). It follows that an average 24 nm λ_{LSPR} shift is quite unprecedented and surprising considering an increase of only 0.26 nm in the thickness of the local dielectric hydrocarbon layer around TNPs. According to the calibration curve we developed (FIG. 1D) for Δλ_{LSPR} as a function of dielectric layer thickness, a 0.26 nm change in the thickness should provide a APLSPR value of only 5.4 nm. Therefore, we hypothesize that a large portion of observed Δλ_{LSPR} is caused by dipole-dipole interactions between the zwitterionic MC form and attached TNPs. We calculated the dipole moment of SP-UT and MC-UT molecules to be 2.77 and 23.78 D, respectively. This large change in dipole moment to a highly polar MC system can significantly change the local refractive index around TNPs. Therefore, the difference in dipole moment between SP and MC could manipulate the plasmonic properties of the system such that the plasmonic TNP and the dipolar organic molecular shell could more strongly couple in the MC form. A simple description of induced polarization and dipolar contribution is given by the classical electrodynamic interaction, as described by Eq. 1.

$$\frac{κ - 1}{κ + 2} = \frac{N}{3 {ε}_{0}} ( {α}_{0} + \frac{{μ}^{2}}{3 kT} ) \qquad ( 1 )$$

Here, κ is the dielectric constant, co is the permittivity of free space, N is the number of molecules, α₀ is the molecular polarizability (induced dipole moment), μ is the permanent dipole moment, k is the Boltzmann constant, and T is the temperature. For simplicity, molar mass and density of surface functionalized molecules were avoided. In the absence of high magnetic polarizability at visible frequency for Au, κ=n², where n is the index of refraction, leading to Lorentz and Lorenz relation (Eq. 2).

$${n}^{2} = \frac{9 {ε}_{0} KT + 2 N ( 3 {α}_{0} KT + {μ}^{2} )}{9 {ε}_{0} KT - N ( 3 {α}_{0} KT + {μ}^{2} )} \qquad ( 2 )$$

Therefore, an increase in permanent dipole moment would increase the overall n value at a constant temperature. Taken together, the MC form with a 23.78 D dipole moment would change the n value significantly more in comparison to SP with 2.77 D. This nanostructure-molecule dipole-dipole interaction can be compared to near-field dipole-dipole interactions between two adjacent nanostructures that result in a LSPR red-shift. We should also acknowledge that electrodynamic coupling is a multivariable interaction, which can be influenced by the induced dipole moment of the organic ligand shell and the spatial organization of both TNPs and their surface ligands. Herein, we considered only the simplest and most important permanent dipole-dipole interactions.

Eq. 3 below suggests a linear relationship between λ_{LSPR} and n. Here λ_{LSPR} and λₚ are LSPR peak wavelength and bulk plasma frequency of the metal, respectively.

$${λ}_{LSPR} = {λ}_{p} ( \sqrt{2 {n}^{2} + 1} ) \qquad ( 3 )$$

Therefore, a change in the dipole moment of the nanostructure surface-bound organic ligand shell can influence the λ_{LSPR}, as observed in our system between SP and MC. Nevertheless, to the best of our knowledge, this is the first example in which photo-isomerization of SP to MC is observed on a nanometer-scale, solid-state device construct by monitoring the LSPR property, except those reported for azobenzene and rotaxane systems, but this current study shows a superior LSPR response. Taken together, the reversible change in LSPR response and high stability of our molecular switch-engineered nanosystem is important for the of design light-controlled plasmonic-based adaptable biosensors. Due to their highest LSPR response, we used 75:25 mixed SP-UT:UT SAMs for our solution biosensing application as described below.

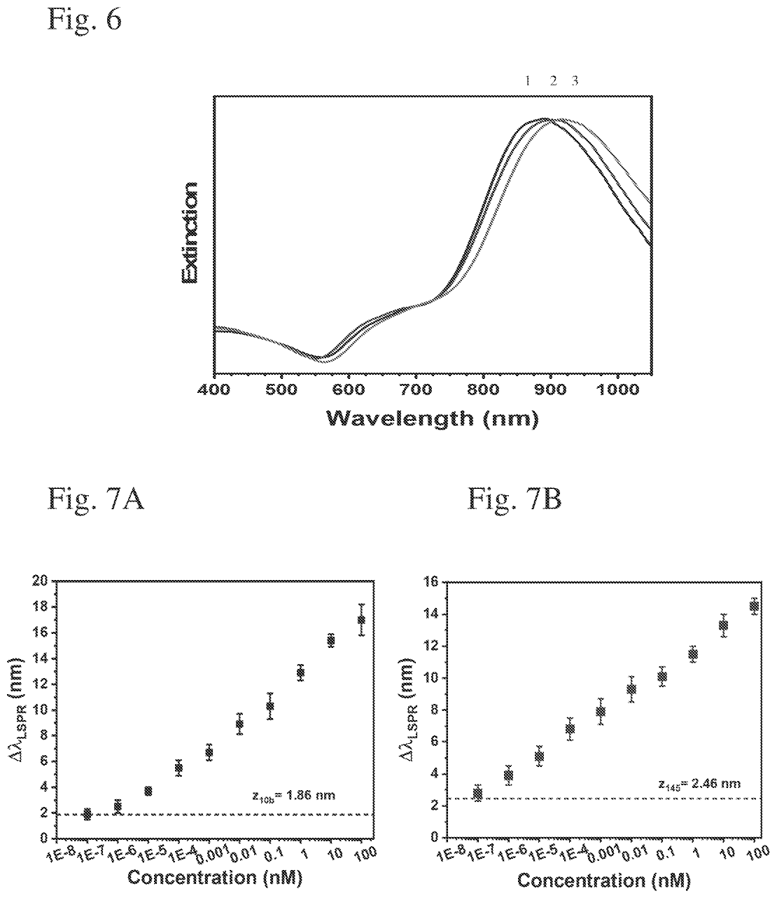

Modern univariant “gold standard” bioanalytical techniques (RT-PCR, microarray, sequencing, ELISA, etc.) almost exclusively operate in a discrete format unable to assay different types of biomolecules using a single instrument. For example, RT-PCR is capable of assaying nucleic acids but not proteins, whereas ELISA only assays proteins but not RNAs. In contrast, as shown in FIG. 1A, we designed a novel nanoplasmonic biosensor that has the ability for highly sensitive measurement of microRNAs and proteins in an identical biosensor construct by simply exchanging the attached receptor molecules by light exposure. We selected these two types of biomolecules because they are excellent biomarkers for early detection of various cancers in liquid biopsies. Numerous studies have shown that microRNA-10b and -145, and protein NuMA are highly specific biomarkers for bladder cancer from two different biological fluids (plasma and urine). FIG. 3A illustrates the UV-Vis extinction spectra of Au TNPs during different functionalization steps in which a receptor (-ssDNA) bound to the zwitterionic MC form (FIGS. 1C, and 1E) is designated as a microRNA “nanoplasmonic biosensor”. Incubation in an aqueous 10 μM -ssDNA-10b solution provides an ˜21 nm λ_{LSPR} red-shift, which is the result of electrostatic adsorption of -ssDNA-10b onto the zwitterionic MC. This shift is mostly from an increase in the local refractive index of the TNPs. The change in Δλ_{LSPR} value is significant considering the molecular weight (MW) of -ssDNA-10b is 7.3 kDa and it is ˜2.92 nm away from the TNP surface. We also believe that the nucleic acid dipoles in -ssDNA may in some manner interact with the MC dipoles and together influence the λ_{LSPR} as a consequence of dipole-dipole interaction. To investigate this interaction further, we performed a control experiment by incubating the same MC-UT modified sensor in a 10 μM aqueous solution of sodium poly (acrylate) (PAA, MW=15 kDa). The negatively charged oxygen pendant group in the polymer backbone mimics the -ssDNA charge but the bases are lacking. We observe a Δλ_{LSPR} of ˜5 nm which is in agreement with the loss of a dipole effect. As illustrated in FIG. 3B, we observe a strong binding affinity between the zwitterionic MC surface and -ssDNA-10b with equilibrium binding dissociation constant (K_{d}) of 106 nM, as determined from the Langmuir isotherm. In a separate control experiment, incubation of 75:25 mixed SAM of SP-UT and UT modified TNPs in a PBS buffer solution of 10 μM -ssDNA-10b does not provide any detectable λ_{LSPR} shift, suggesting -ssDNAs only adsorbs onto the charged MC surface due to electrostatic interaction and not on the neutral SP surface. Finally, incubation of the nanoplasmonic biosensor designed for microRNA-10b detection displays an additional 15 nm λ_{LSPR} red-shift (FIG. 3A). Extinction spectra relevant to microRNA-145 detection are provided in the Supporting Information (FIG. 6). The limit of detections (LODs) for microRNA-10b and 145 are 1.87 and 1.50 femtomolar (fM), respectively (FIG. 3C). The LODs for these microRNAs are in the attomolar (aM) range in PBS buffer (without human plasma) (FIGS. 7A & 7B and Table 3).

TABLE 3
Calibration curve equations and limits of
detections for biomarkers in PBS buffer.

| | Calibration | R² | Blank | Z value | |
|---|---|---|---|---|---|
| Biomarker | Curve Equation | value | (nm) | (nm) | LOD |
| microRNA- | y = 1.71774 | 0.974 | 0.90 ± 0.3 | 1.86 | 421.9 aM |
| 10b in PBS | log(x) + 12.81025 | | | | |
| buffer | | | | | |
| microRNA- | y = 1.29515 | 0.998 | 0.96 ± 0.5 | 2.46 | 68.6 aM |
| 145 in PBS | log(x) + 11.73835 | | | | |
| buffer | | | | | |
| NuMA in | y = 1.45785 | 0.977 | 1.1 ± 0.4 | 2.37 | 137.0 aM |
| PBS buffer | log(x) + 12.37634 | | | | |

Although LSPR sensitivity can be enhanced by selecting larger aspect ratio nanostructures that display an λ_{LSPR} at longer wavelengths (>800 nm), with an increasing aspect ratio plasmonic line-width increases, which broadens the LSPR peak and increases non-specificity in biosensing. Herein, we have shown that functionalizing Au TNPs with appropriate SAMs bearing zwitterion molecules can shift the λ_{LSPR} to longer wavelengths (enhancing sensing efficiency) without physically changing the aspect ratio of a nanostructure. We also examined the specificity of our nanoplasmonic biosensor because it is critically important in clinical diagnostics. This is more important in the current construct, since all the interactions are electrostatic and any plasma microRNAs can adsorb on unoccupied MC sites providing false response. The nanoplasmonic biosensor designed to detect microRNA-10b was incubated in a 10% plasma solutions containing microRNA-145, -143, -490-5p, and -96 (10.0 nM/microRNA), which are found to be over and/or underexpressed in bladder cancer, and after washing the biosensor and measuring the extinction spectrum provides Δλ_{LSPR}˜1.4 nm (FIG. 3D), which could be due to transient adsorption of some microRNAs and/or instrumental noise.

The adaptability of the present biosensor is further illustrated (see FIGS. 1D and 1F) by detecting and quantifying NuMA protein, which is a Federal Drug Administration (FDA)-approved biomarker for bladder cancer. The level of NuMA protein is generally measured in urine. We used the reversible photoswitching property of our 75:25 mixed SP-UT and UT SAM-functionalized Au TNPs to demonstrate the adaptability of our nanoplasmonic biosensor. As shown in FIG. 3E, incubation of mixed MC-UT and UT SAM-functionalized Au TNPs in a 100 ng/ml of anti-NuMA solution provides an LSPR red shift (Δλ_{LSPR}) of 32nm. Then after rinsing the biosensor followed by incubation in 100 nM NuMA in 10% urine solution there is an additional 15 nm red-shift. Although the Δλ_{LSPR} values found for -ssDNA-10b/145 and anti-NuMA adsorption onto the biosensor MC surface are significantly different due to substantially different overall geometric of binding, the degree of allowed electrostatic and dipolar interactions and MWs, the Δλ_{LSPR} values are surprisingly comparable for microRNA-10b/145 and NuMA binding to their respective biosensors. One would expect a larger Δλ_{LSPR} value upon NuMA adsorption onto anti-NuMA than 15 nm because of the large size and higher molecular weight protein. We determined the interaction between anti-NuMA and the zwitterion MC surface is strong with K_{d} of 5.05 PM (FIG. 3F). We believe that the bulky nature of anti-NuMA keeps the NuMA further away from the biosensor surface, resulting in a lower LSPR response. Moreover, steric crowding is another factor that may not allow many anti-NuMA to adsorb onto the MC surface, and thus the concentration of bound NuMA is lower. Another possibility is that proteins have multiple charge patches that could nullify some of the dipole interactions. Nevertheless, the LODs for NuMA in 10% urine and pure PBS buffer are determined to be 5.0 fM (0.15 pg/mL) and 130 aM (4.25 fg/mL), respectively, (see FIG. 3G and Table 4).

TABLE 4
Calibration curve equations and limits of detections
for biomarkers in human biofluids.

| | Calibration | R² | Blank | Z value | |
|---|---|---|---|---|---|
| Biomarker | Curve Equation | value | (nm) | (nm) | LOD |
| microRNA- | y = 1.54152 | 0.994 | 0.98 ± 0.4 | 2.08 | 1.87 fM |
| 10b in | log(x) + 10.91023 | | | | |
| plasma | | | | | |
| microRNA- | y = 1.26371 | 0.997 | 0.97 ± 0.3 | 1.87 | 1.50 fM |
| 145 in | log(x) + 9.22914 | | | | |
| plasma | | | | | |
| NuMA in | y = 1.57453 | 0.994 | 1.2 ± 0.5 | 2.70 | 5.0 fM |
| 10% urine | log(x) + 11.04575 | | | | |

Finally, we tested the specificity of our biosensors for protein detection by incubating them in a 100 nM prostate specific antigen (PSA) solution, and a negligible ALLSPR of 1.1 nm is observed (FIG. 3H). This result is significant and suggests that although our biosensing mechanism relies on charge-charge interaction between MC-receptor and receptor-analyte molecules, under our optimized fabrication strategy, non-specific adsorption of unwanted biomolecules, and thus false positive responses can be avoided.

### EXAMPLE 3

### Regeneration of the Biosensor

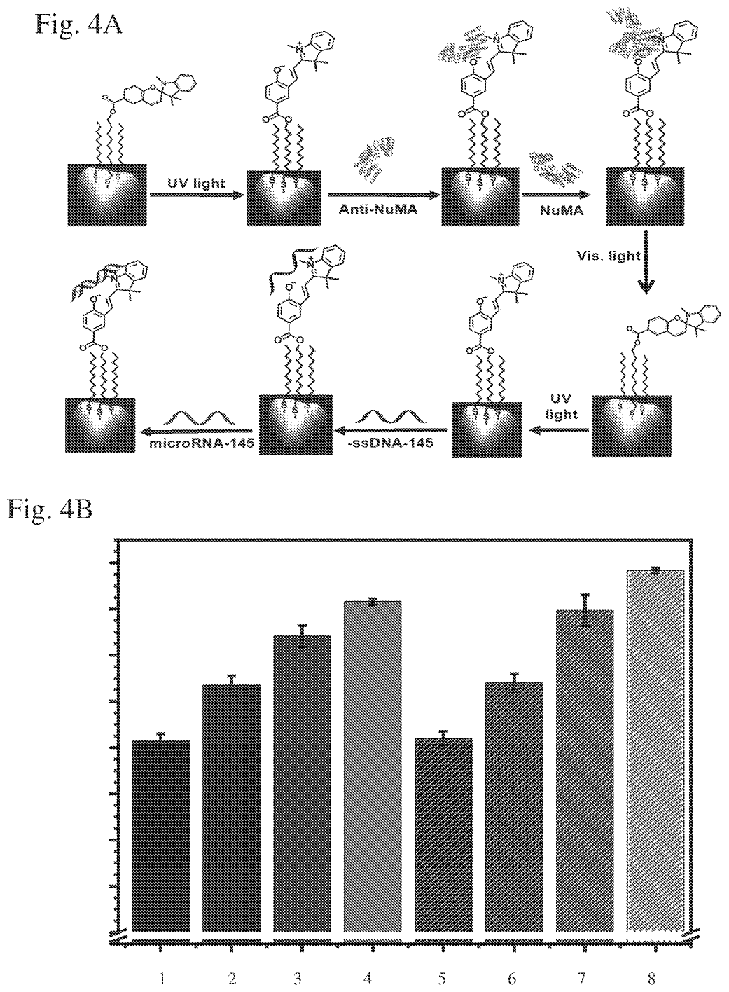

One of the main accomplishments of the present disclosure relates to the demonstration of regenerative testing capabilities for different biomarkers (adaptability), e.g., first as a microRNA assay and then regenerating the biosensor to measure proteins, all using the same device and identical signal output. Biosensor regeneration is also an important aspect in lowering costs, particularly important in low-income countries. Our strategy is less labor intensive than PCR and ELISA, offers excellent protocol verification, and provides excellent biosensor calibration and standardization. To test regeneration, as schematically illustrated in FIG. 4A, the NuMA biosensor was prepared and it's λ_{LSPR} value was determined in the presence of 100 nM NuMA resulting in a Δλ_{LSPR} of 16.7 nm (FIG. 4B). The biosensor was then rinsed with 2.5 M PBS buffer to weaken the protein-protein and protein MC interactions through charge screening, rinsed in water, dried under N₂ flow, and finally exposed to visible light for 10 min. The last step causes the conversion of the MC to SP form, regenerating the original mixed SP-UT and UT SAM-functionalized Au TNPs, which then display an λ_{LSPR} at 864 nm implying removal of the bound receptor/analyte pair. This value is within the range of freshly prepared, 75:25 mixed SP-UT and UT SAM-functionalized Au TNPs (FIG. 1D). Next, we exposed this substrate to UV light to generate the activated MC form, which was incubated in 10 μM -ssDNA-145 as the new receptor. After washing, the biosensor was incubated with 100 nM microRNA-145 in 10% human plasma. The biosensor sensitivity after regeneration and assay of microRNA-145 is comparable to freshly prepared biosensors. Although, the interaction between the zwitterion MC form and receptor molecules (either -ssDNA-10b/145 or anti-NuMA) are relatively strong, the activation energy for SP-MC isomerization is ˜50 kJ/mol in polar solvents. We believe washing the biosensors with high concentration of electrolyte solution and this relative low activation energy allow the biosensors to regenerate to the non-receptor binding SP motif.

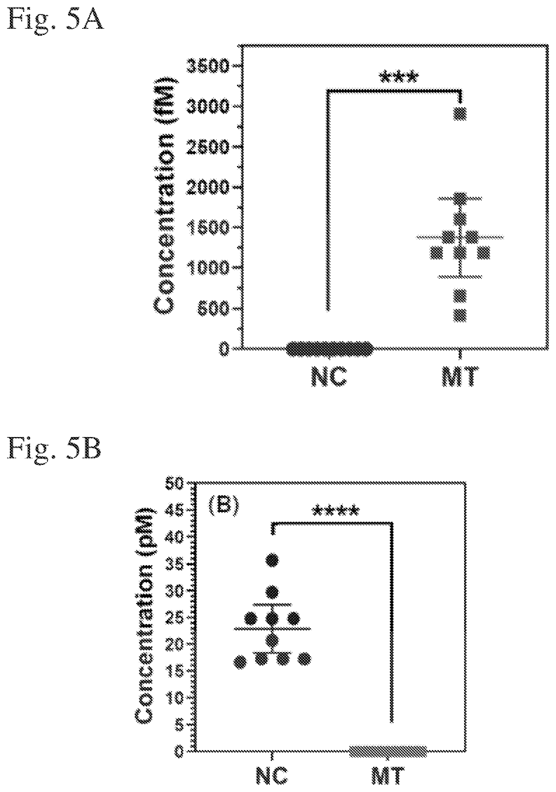

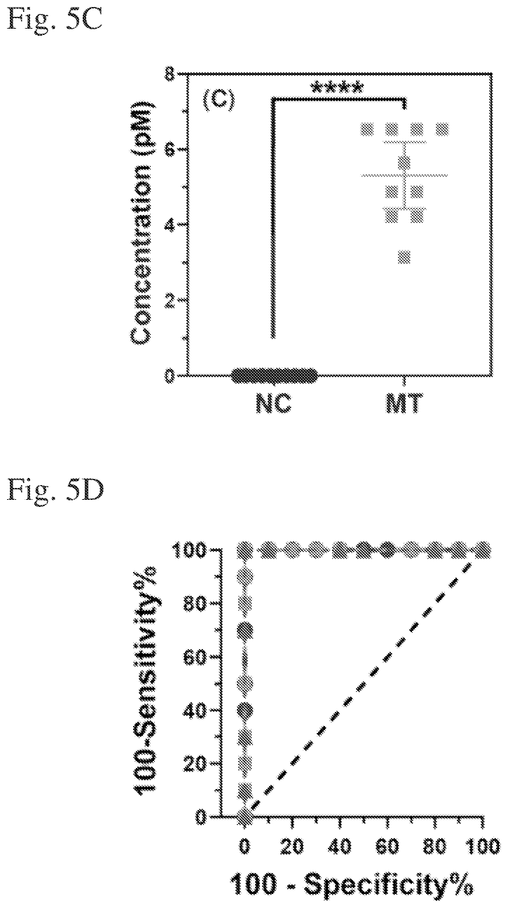

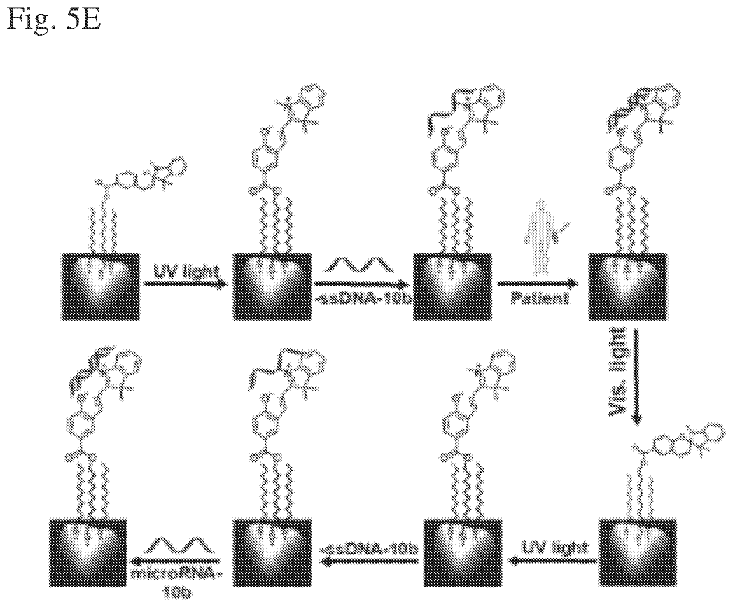

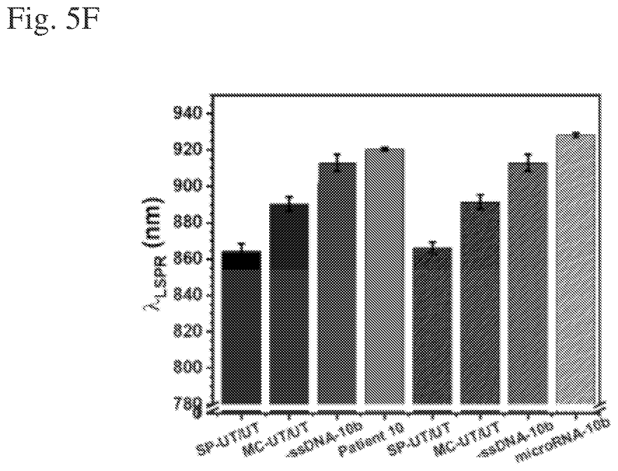

To evaluate the performance of our adaptable nanoplasmonic biosensor assay strategy for clinical diagnosis, real world clinical samples from 10 metastatic (MT) bladder cancer patients were analyzed. We assayed 10 plasma samples to detect and quantify both microRNA-10b and microRNA-145. It is important to mention that microRNA-10b and microRNA-145 are oncogenic and tumor-suppressor biomolecules, respectively, and the microRNA-10b level increases whereas the microRNA-145 level decreases in cancer patient with respect to healthy subjects (normal control, NC). As shown in FIGS. 5A-5B, both microRNAs are highly selective biomarkers to distinguish cancer patients (n=10) from NC (n=10). However, tumor suppressor microRNA-145 is found to be a more selective biomarker with a p-value <0.0001. We also quantified NuMA protein for the above cancer patients by analyzing urine samples. FIG. 5C presents a p-value of <0.0001 for MT cancer patient versus NC. Receiving operating characteristic (ROC) analysis reveals that our newly designed nanoplasmonic biosensor is highly selective and can discriminate between the NC versus the disease group with an area under the curve (AUC) of 1.0 (FIG. 5D). Importantly, this is the first example where both nucleic acid and protein biomarkers are assayed utilizing a single instrument, reducing time-lag dramatically and improving diagnosis by faster data correlation. Our detection method can also be used for excellent protocol verification and quality control via a biosensor regeneration study. As schematically represented in FIG. 5E, we detected and quantified microRNA-10b from a patient plasma and regenerated the biosensor SP motif, which was then used to analyze a standard of 100 nM microRNA-10b. The LSPR data presented in FIG. 5F show that even after patient sample analysis, our nanoplasmonic biosenors can be regenerated and to detect microRNAs with a similar efficiency as freshly prepared biosensors.

Utilizing the unique plasmonic properties of Au TNPs that enhance surface-enhanced Raman scattering (SERS), we experimentally analyzed mixed SP-UT:UT and MC-UT:UT SAM-modified Au TNPs with Raman spectroscopy. The SERS spectrum of SP-UT (FIG. 8, left panel) consists of Raman stretches at 1720, 1574, 1519, 1428, 1355, 1297, 1142, 1116,1080, 1020, 951, and 820 cm⁻¹. The Raman peaks at 1519/1355, 1142, and 951 cm⁻¹ represent C—N asymmetric, N—CH₃ aromatic, and O—C—N stretches, respectively. Furthermore, the peak at 1720 cm⁻¹ represents C═O and the peak at 820 cm⁻¹ represents C—O aromatic stretches, respectively. Additional peaks including those at 1574,1297, 1080, and 1020 cm⁻¹ represent indoline and chromene moieties. Peaks at 1428 and 1116 cm⁻¹ represent CH₂ stretching from the UT chain. As shown in FIG. 8 (right panel), new Raman stretches appear at 1655, 1590, 1525, 1194, and 715 cm⁻¹. Unique to MC, the C═C trans alkene peak appears at 1655 cm⁻¹. Furthermore, the new C═N stretch and C—N—C scissoring caused by the ring opening of SP to MC appear at 1525 and 715 cm⁻¹, respectively.

Utilizing the highly polar form of a photo-isomerizable molecular switch, i.e., SP(MC), chemically attached onto Au TNPs via SAMs, the LSPR response can be modulated by nonradiative dipole-dipole interactions without changing the physical dimensions of the nanostructures and surrounding dielectric components. Moreover, using the extraordinary range of structure and properties of the SP based system, we have constructed a responsive nanosystem, which has now been used for an ultrasensitive assay of both nucleic acids and proteins adaptively with identical workflow and highly selective device readout. As a proof-of-concept, we have also shown that our sensing approach can be applied to detect bladder cancer biomarkers from different human specimens. Taken together, this inexpensive but highly sophisticated technology can be used for rapid clinical setting characterization of other cancers, thus providing a new paradigm in early detection of several biomarkers from different biofluids for non-invasive “liquid biopsies.”

1. An adaptable nanoplasmonic biosensor for specific detection of target proteins and nucleic acids, said biosensor comprising
a localized surface plasmon resonance (LSPR) chip having an affixation surface and a functional surface;
a functionalized solid support; and
an LSPR antennae comprising a light inducible isomerizable compound, wherein
said affixation surface of the LSPR chip is covalently linked to said solid support, and said LSPR antennae is linked to said functional surface of the LSPR chip.

2. The biosensor of claim 1 wherein the LSPR chip is a metal comprising triangular nanoprism, wherein the metal is selected from the group consisting of gold, silver, copper, palladium, aluminum, or a combination thereof, and said LSPR chip is covalently linked to said solid support via a plurality of spacer molecules that comprise a poly-ethylene glycol moiety, an alkyl moiety, or a combination thereof.

3. The biosensor of claim 2 wherein the LSPR chip is a gold triangular nanoprism (Au TNP) and said spacer molecules comprise a first end bound to the solid support and a second end comprising a functional group, optionally a thiol, that forms a covalently bond to a group located on the LSPR chip.

4. The biosensor of claim 2 wherein said LSPR antennae are covalently linked to said functional surface of the LSPR chip via a plurality of alkylthiolate spacer molecules, optionally wherein the alkylthiolate spacer molecules are nonanethiol or undecanethiol spacer molecules.

5. The biosensor of claim 4 wherein the Au TNP has an average edge-length of between 30 and 50 nm.

6. The biosensor of claim 5 wherein the solid support is substantially transparent to electromagnetic radiation having a wavelength between 100 nm and 700 nm.

7. The biosensor of claim 1 wherein the light inducible isomerizable compound forms a zwitterion upon exposure to UV light and said light inducible isomerizable compounds are covalently linked to said functional surface of the LSPR chip via a plurality of alkylthiolate spacer molecules, optionally wherein the alkylthiolate spacer molecules are undecanethiol spacer molecules.

8. The biosensor of claim 7 wherein the light inducible isomerizable compound has the general structure of 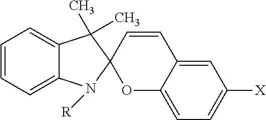
wherein R is selected from the group consisting of —(CH₂)ᵤN⁺(CH₃)₃, wherein n is an integer selected from 1 to 6, and
X is —NO₂, —CONH₂ or —COOH.

9. The biosensor of claim 7 wherein the light inducible isomerizable compound has the structure of

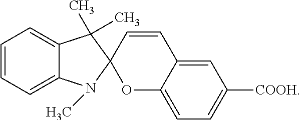 [SMILES: CN1c2ccccc2C(C)(C)C12C=Cc1cc(C(=O)O)ccc1O2]

10. The biosensor of claim 7 wherein the LSPR antennae comprises the structure: 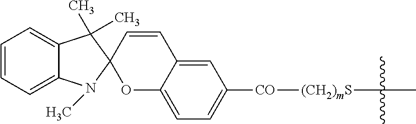
wherein m is an integer selected from 9 to 13.

11. The biosensor of claim 10 wherein m is 11 (SP-UT).

12. The biosensor of claim 11 further comprising a plurality of undecanethiol (UT) polymers linked to said functional surface of the LSPR chip.

13. The biosensor of claim 12 wherein the LSPR chip comprises a 75%:25% mixture of SP-UT and UT linked to said functional surface of the LSPR chip.

14. A method of detecting the presence of a first and second analyte in a biological sample through the use of a single device utilizing an identical signal output for the detection of both the first and second analyte, said method comprising
a) providing a nanoplasmonic biosensor according to claim 1;
b) exposing the biosensor to UV light to induce photoisomerization of said isomerizable compound and produce the zwitterion form of said compound;
c) contacting the zwitterion form of said compound with a first ligand that specifically binds to said first analyte, wherein the first ligand electrostatically binds to the zwitterion form of said compound to form a first ligand complex;
d) contacting said first ligand complex with a biological sample;
e) conducting LSPR analysis of said nanoplasmonic biosensor after step d);
f) optionally rinsing said first ligand complex with a buffer to promote charge screening to disrupt binding of the first ligand to the zwitterion form of said compound;
g) exposing the biosensor to visible light to regenerate the non-zwitterion form of said compound and rinsing said biosensor to remove unbound material;
h) exposing the biosensor to UV light to induce photoisomerization of said isomerizable compound and produce the zwitterion form of said compound;
i) contacting the zwitterion form of said compound with a second ligand that specifically binds to said second analyte, wherein said second analyte electrostatically binds to the zwitterion form of said compound to form a second ligand complex;
j) contacting said second ligand complex with a biological sample;
k) conducting LSPR analysis of said nanoplasmonic biosensor after step j).

15. The method of claim 14 wherein said non-zwitterion form of said compound comprises the structure of
of 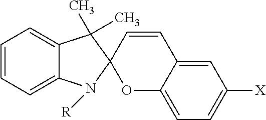
wherein R is selected from the group consisting of —(CH₂)ₙN⁺(CH₃)₃, wherein n is an integer selected from 1 to 6, and
X is —NO₂, —CONH₂ or —COOH; and
said zwitterion form of said compound comprises the structure of 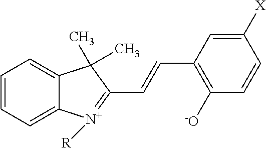
wherein R is selected from the group consisting of —(CH₂)ₙN⁺(CH₃)₃, wherein n is an integer selected from 1 to 6, and
X is —NO₂, —CONH₂ or —COOH.

16. The method of claim 15 wherein the LSPR chip is a gold triangular nanoprism (Au TNP), said light inducible isomerizable compounds are covalently linked to said functional surface of said Au TNP via a plurality of undecanethiol spacer molecules, wherein said functional surface of said Au TNP further comprises a plurality of undecanethiol (UT) polymers linked to said functional surface of the LSPR chip.

17. The method of claim 15 wherein the steps of conducting LSPR analysis comprises measuring an absorption spectrum of the LSPR antenna, the absorption spectrum having a peak wavelength; and determining the presence or quantity of the first and second analyte in said sample based on the peak wavelength.

18. The method of claim 17, wherein the method of
detecting the presence of the first analyte in said sample comprises measuring a first absorption spectrum of the LSPR antenna after step c) and measuring a second absorption spectrum of the LSPR antenna after step d) and determining the difference between the peak wavelength of the first and second measured absorption spectrum of the LSPR antenna; and
detecting the presence of the second analyte in said sample comprises measuring a third absorption spectrum of the LSPR antenna after step i) and measuring a fourth absorption spectrum of the LSPR antenna after step j) and determining the difference between the peak wavelength of the third and fourth measured absorption spectrum of the LSPR antenna.

19. The method of claim 15 wherein the first analyte is a protein, the second analyte is a nucleic acid, the first ligand is a protein receptor that specifically binds to said protein and the second ligand is a nucleic acid sequence that specifically binds to said second analyte nucleic acid.

20. The method of claim 15 wherein the first analyte is a first RNA, the second analyte is a second RNA, the first ligand is a first nucleic acid sequence that specifically binds to said first RNA and the second ligand is a nucleic acid sequence that specifically binds to said second RNA, wherein the first and second RNAs have difference nucleic acid sequences.
